(19) United States
(12) Patent Application Publication
(10) Pub. No.: US 2023/0111847 A1
(43) Pub. Date: Apr. 13, 2023

(54) ANATOMIC KNEE PROSTHESIS AND DESIGNING METHOD

(71) Applicant: SYMBIOS ORTHOPÉDIE S.A., Yverdon-les-Bains (CH)

(72) Inventors: Vincent LECLERCQ, Echandens (CH); Florent PLÉ, Préverenges (CH)

(21) Appl. No.: 17/794,334

(22) PCT Filed: Jan. 22, 2021
(86) PCT No.: PCT/IB2021/050496

§ 371 Date: Jul. 21, 2022

Related U.S. Application Data
(60) Provisional application No. 62/964,170, filed on Jan. 22, 2020.
(60) Provisional application No. 62/964,182, filed on Jan. 22, 2020.
(60) Provisional application No. 63/294,942, filed on Dec. 30, 2021.

(51) Int. Cl. A61B 34/10; A61F 2/38

(52) U.S. Cl. CPC A61B 34/10; A61F 2/389; A61F 2/3877; A61B 2034/104; A61F 2002/4633

(57) ABSTRACT

A knee prosthesis and a method of selecting for a particular patient a knee prosthesis from an inventory of available knee prosthesis or from 3D knee prosthesis models is provided. The method includes the steps of (a) parameterizing a knee prosthesis according to well-defined and independent knee joint compartments, (b) generating a large number knee shapes in the form of 3D knee prosthesis models which reproduce the 3D shape asymmetries of each individual knee, thereby enabling the replication of the knee motion of essentially any patient by generating shapes which vary the shape parameters (surface and dimension) of at least one of those compartments and storing these 3D knee prosthesis models in a database, and (c) studying the patient’s pathology and developing pre -pathological knee prosthesis criteria matching the patient’s needs. Using a planning algorithm, a suitable knee prosthesis may be selected among an inventory of existing knee prostheses or the large number of knee shapes, the prosthesis or shape selected best meeting the patient’s needs as determined by the study.

### CROSS REFERENCE TO RELATED APPLICATIONS

This application claims the benefit of U.S. Provisional Application No. 62/964,170, filed Jan. 22, 2020 and U.S. Provisional Application 62/964,182, filed Jan. 22, 2020, the content of the entirety of which is explicitly incorporated herein by reference and relied upon to define features for which protection may be sought hereby as it is believed that the entirety thereof contributes to solving the technical problem underlying the invention, some features that may be mentioned hereunder being of particular importance.

### IDENTIFICATION OF PARTIES CONCERNED

The Applicant of the present intellectual property matter is Symbios Orthopedic S.A. of Switzerland. The inventor(s) of the invention described in this patent documentation are Vincent LECLERCQ, French citizen of Echandens, Switzerland, and Florent PLE, French citizen, of Preverenges, Switzerland. Other inventors may be added at the time of filing of a regular application. At the time of filing, John B. Moetteli and the firm Da Vinci Partners LLC of Switzerland represent the Applicant.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

Today’s knee prostheses have been optimized in terms of industrial production and therefore standardization. Such prostheses of the prior art have been found satisfying in terms of life duration (+93% after 15 years) but low customer satisfaction (only 70%). Due to this situation, a number of patients who would benefit from a prosthesis delay the operation, fearing the risk of discomfort or pain, and end up with a more severe condition, eventually leading to more serious treatment needs. The chronic discomfort and the pain following the knee joint replacement operation are notably generated by the different behavior of standardized prosthesis compared to the anatomical behavior of an individual’s original knee joint, which is specific to each individual patient. When a first knee is replaced with a prosthesis, and the patient does not have the second knee replaced at the same time, using a standard knee prosthesis of the prior art may generate an unbalanced behavior between the replaced knee joint and the original knee joint, resulting in an accelerated aging and/or deformation of the non-replaced knee joint, eventually leading to the necessity of prematurely replacing the second knee joint.

What is needed therefore is a knee prosthesis that can replace the natural knee joint of a patient in an individually adapted anatomic manner, so as to minimize the risk of discomfort or pain, and without generating collateral damage to the other knee joint.

### SUMMARY OF THE INVENTION

A knee prosthesis and a method of selecting from an inventory of available knee prostheses or from 3D knee prosthesis model for a particular patient is provided. The method includes the steps of (a) parameterizing a knee prosthesis according to well-defined and independent knee joint compartments, (b) generating a large number knee shapes in the form of 3D knee prosthesis models which reproduce the 3D shape asymmetries of each individual knee, thereby enabling the replication of the knee motion of essentially any patient by generating shapes which vary the shape parameters (surface and dimension) of at least one of those compartments and storing these 3D knee prosthesis models in a database, and (c) studying the patient’s pathology and developing pre-pathological knee prosthesis criteria matching the patient’s needs. A suitable knee prosthesis may be selected among an inventory of existing knee prostheses or the large number of knee shapes, the prosthesis or shape selected best meeting the patient’s needs as determined by the study.

In a further embodiment, the method includes consideration of the articulation of the patella in selecting a suitable 3D model.

The present invention provides a knee prosthesis that is much better adapted to the need of a given individual patient compared to the knee prostheses of the current state of the art, and results in a lower risk of chronical pains and/or discomfort.

### BRIEF DESCRIPTION OF THE DRAWINGS

The attached drawings represent, by way of example, different embodiments of the invention.

FIG. 1A is a schematic, three-quarter view of a knee joint.

FIG. 1B is a schematic, medial view of a knee joint.

FIG. 2A is a schematic, front view of HKA (Hip-Knee-Ankle) normal alignment, also referred to as “neutral alignment” or “straight alignment”.

FIG. 2B is a schematic, front view of HKA varus (or bowleg) alignment.

FIG. 2C is a schematic, front view of HKA valgus (or knock knee) alignment.

FIG. 3 is a flow chart of a method according to the invention.

FIG. 4 is a flow chart of first variant of the method according to the invention.

FIG. 5 is a flow chart of a second variant of the method according to the invention.

FIG. 6 is a flow chart of a third variant of the method according to the invention.

FIG. 7A is a front view of the femur.

FIG. 7B is a top view of the femur.

FIG. 8A is a front view of a knee articulation while extended.

FIG. 8B is a front view of a knee articulation while bent.

FIG. 9A is an ISO view of the femoral implant of a knee prosthesis according the invention.

FIG. 9B is a frontal view of the femoral implant of a knee prosthesis according the invention.

FIG. 9C is a sagittal view of the femoral implant of a knee prosthesis according the invention.

FIG. 10A is a frontal view of the knee prosthesis of the invention.

FIG. 10B is an axial view of the knee prosthesis of the invention.

FIG. 10C is a sagittal view of the knee prosthesis of the invention.

FIG. 11A is a frontal view of a knee prosthesis of the prior art.

FIG. 11B is a frontal view of the knee prosthesis of the invention.

FIG. 11C is an axial view of a knee prosthesis of the prior art.

FIG. 11D is an axial view of the knee prosthesis of the invention,

FIG. 12A is a coronal view of a knee prosthesis of the invention in the case of constitutional femur neutral (symmetrical distal condyles), in extension (symmetrical distal condyles).

FIG. 12B is a transverse view of a knee prosthesis of the invention with symmetrical posterior condyles, in flexion (symmetrical posterior condyles).

FIG. 12C is a transverse view of a knee prosthesis of the invention in flexion, where the posterior lateral condyle is shorter than the posterior medial condyle (internally rotated posterior condyles).

FIG. 12D is a transverse view of a knee prosthesis of the invention in flexion, where the posterior lateral condyle is longer than the posterior medial condyle (externally rotated posterior condyles).

FIG. 12E is a transverse view of a knee prosthesis of the invention in flexion, where an angle is opening from the posterior lateral condyle to the posterior medial condyle (internally rotated posterior condyles).

FIG. 12F is a transverse view of a knee prosthesis of the invention in flexion, where an angle is opening from the posterior medial condyle to the posterior lateral condyle (externally rotated posterior condyles).

FIG. 12G is a coronal view of a knee prosthesis of the invention in the case of constitutional femur neutral (symmetrical distal condyles) in extension (symmetrical distal condyles).

FIG. 12H is a coronal view of a knee prosthesis of the invention in the case of constitutional femur valgus in extension, where the distal lateral condyle is shorter than the distal medial condyle.

FIG. 12I is a coronal view of a knee prosthesis of the invention in the case of constitutional femur varus in extension, where the distal lateral condyle is longer than the distal medial condyle.

FIG. 12J is a coronal view of a knee prosthesis of the invention in the case of constitutional femur valgus in extension, where an angle opens from the distal lateral condyle to the distal medial condyle.

FIG. 12K is a coronal view of a knee prosthesis of the invention in the case of constitutional femur varus in extension, where an angle opens from the distal medial condyle to the distal lateral condyle.

FIG. 13A is a coronal view of a knee prosthesis of the invention in extension.

FIG. 13B is a transverse view of a knee prosthesis of the invention in flexion.

FIG. 13C is a sagittal view of the femoral implant of a knee prosthesis according the invention.

FIG. 14A is a cross section view in the coronal plane of a femoral implant of the invention in extension, showing a plane resection.

FIG. 14B is a cross section view in the transverse plane of a femoral implant of the invention in flexion, showing a plane resection.

FIG. 14C is a cross section view in the coronal plane of a femoral implant of the invention in extension, showing an inclined resection.

FIG. 14D is a cross section view in the transverse plane of a femoral implant of the invention in flexion, showing an inclined resection.

FIG. 14E is a cross section view in the coronal plane of a femoral implant of the invention in extension, showing a curved resection.

FIG. 14F is a cross section view in the transverse plane of a femoral implant of the invention in flexion, showing a curved resection.

FIG. 14G is a cross section view in the coronal plane of a femoral implant of the invention in extension, showing an offset resection.

FIG. 14H is a cross section view in the transverse plane of a femoral implant of the invention in flexion, showing an offset resection.

FIG. 14I is a cross section view in the coronal plane of a femoral implant of the invention in extension, showing a double inclined resection.

FIG. 14J is a cross section view in the transverse plane of a femoral implant of the invention in flexion, showing a double inclined resection.

FIG. 14K is a sagittal view of a femoral implant of the invention in extension adapted for a sawed resection.

FIG. 14L is a sagittal view of a femoral implant of the invention in extension adapted for a milled resection.

FIG. 15A is a coronal view of the femoral implant and the tibial insert component of a knee prosthesis of the invention in the case of constitutional femur neutral in extension.

FIG. 15B is a coronal view of the femoral implant and the tibial insert component of a knee prosthesis of the invention in the case of constitutional femur varus in extension, where the distal medial condyle is shorter than the distal lateral condyle.

FIG. 15C is a coronal view of the femoral implant and the tibial insert component of a knee prosthesis of the invention in the case of constitutional femur valgus in extension, where the distal medial condyle is longer than the distal lateral condyle.

FIG. 15D is a coronal view of the femoral implant and the tibial insert component of a knee prosthesis of the invention in the case of constitutional femur valgus in extension, where an angle opens from the distal medial condyle to the distal lateral condyle.

FIG. 15E is a coronal view of the femoral implant and the tibial insert component of a knee prosthesis of the invention in the case of constitutional femur varus in extension, where an angle opens from the distal lateral condyle to the distal medial condyle.

FIG. 16A is a schematic coronal view combining the tibial insert, the tibial tray and the keel of a knee prosthesis of the invention.

FIG. 16B is a schematic coronal view combining the tibial insert, the tibial tray and the keel of a knee prosthesis of the invention, where the keel is oriented at an angle.

FIG. 16C is a schematic coronal view combining the tibial insert, the tibial tray and the keel of a knee prosthesis of the invention, where the tibial bone facing surface and the keel are oriented at different angles, resulting in a thinner medial insert thickness, favouring the knee to be orientated in varus.

FIG. 16D is a schematic coronal view combining the tibial insert, the tibial tray and the keel of a knee prosthesis of the invention, where the tibial bone facing surface and the keel are oriented at different angles, and the distal lateral condyle is shorter than the distal medial condyle.

FIG. 16E is a schematic coronal view combining the tibial insert, the tibial tray and the keel of a knee prosthesis of the invention, where the tibial bone facing surface, the keel and the bicondyle distal tangent are oriented at different angles.

FIG. 17A is a schematic sagittal view combining the tibial insert, the tibial tray and the keel of a knee prosthesis of the invention, showing a J-curve defined by 2 radii.

FIG. 17B is a schematic sagittal view combining the tibial insert, the tibial tray and the keel of a knee prosthesis of the invention, showing a J-curve defined by 2 radii, where the keel is offset towards the anterior part of the tibia.

FIG. 17C is a schematic sagittal view combining the tibial insert, the tibial tray and the keel of a knee prosthesis of the invention, showing a J-curve defined by 2 radii, where the keel is offset towards the posterior part of the tibia.

FIG. 17D is a schematic sagittal view combining the tibial insert, the tibial tray and the keel of a knee prosthesis of the invention, showing a J-curve defined by 3 radii.

FIG. 17E is a schematic sagittal view combining the tibial insert, the tibial tray and the keel of a knee prosthesis of the invention, showing a J-curve defined by 4 radii.

FIG. 17F is a schematic sagittal view combining the tibial insert, the tibial tray and the keel of a knee prosthesis of the invention, showing a J-curve defined by 2 radii, where the keel and the tibial bone facing surface are oriented at an angle.

FIG. 17G is a schematic sagittal view combining the tibial insert, the tibial tray and the keel of a knee prosthesis of the invention, showing a J-curve defined by 3 radii, where the keel and the tibial bone facing surface are oriented at an angle.

FIG. 17H is a schematic sagittal view combining the tibial insert, the tibial tray and the keel of a knee prosthesis of the invention, showing a J-curve defined by 4 radii, where the keel and the tibial bone facing surface are oriented at an angle.

FIG. 17I is a schematic sagittal view combining the tibial insert, the tibial tray and the keel of a knee prosthesis of the invention, showing a J-curve defined by 4 radii, where the keel and the tibial bone facing surface are oriented at an angle, and the keel is offset towards the posterior condyle.

FIG. 18 is a top view of the tibial component of a knee prosthesis of the invention

FIG. 19A is a coronal view of the tibial tray and keel of a knee prosthesis of the invention presenting an orientation angle.

FIG. 19B is a coronal view of the tibial tray and keel of a knee prosthesis of the invention presenting an orientation angle and an offset.

FIG. 19C is a coronal view of the tibial tray and keel of a knee prosthesis of the invention presenting an orientation angle and an offset.

FIG. 19D is a coronal view of the tibial tray and keel of a knee prosthesis of the invention presenting two orientation angles.

FIG. 19E is a coronal view of the tibial tray and keel of a knee prosthesis of the invention presenting two orientation angles.

FIG. 20A is a coronal view of the tibial tray and keel of a knee prosthesis of the invention adapted for a bone resection presenting an angle on the medial side.

FIG. 20B is a coronal view of the tibial tray and keel of a knee prosthesis of the invention adapted for a bone resection presenting an angle on the lateral side.

FIG. 20C is a coronal view of the tibial tray and keel of a knee prosthesis of the invention adapted for a bone resection presenting a step on the medial side.

FIG. 20D is a coronal view of the tibial tray and keel of a knee prosthesis of the invention adapted for a bone resection presenting a step on the lateral side.

FIG. 21A is a coronal view of the femoral implant and the patella component of a knee prosthesis of the invention in the case of constitutional femur neutral.

FIG. 21B is a coronal view of the femoral implant and the patella component of a knee prosthesis of the invention in the case of constitutional femur varus, where the distal lateral condyle is shorter than the distal medial condyle.

FIG. 21C is a coronal view of the femoral implant and the patella component of a knee prosthesis of the invention in the case of constitutional femur valgus, where the distal lateral condyle is longer than the distal medial condyle.

FIG. 22A is a coronal view of the patella component of a knee prosthesis of the invention.

FIG. 22B is a sagittal view of the patella component of a knee prosthesis of the invention.

FIG. 23 is a transverse view of the patella component of a knee prosthesis of the invention.

FIGS. 24A to 24F is a flow chart of another variant of the method according to the invention.

FIG. 25A is a flow chart describing different routines of the invention.

FIG. 25B is a sketch/representation of a knee prothesis of the invention.

FIGS. 26A to 26D are describing femoral component compartmentalization and parameterization.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms “first”, ‘second’, and the like are used herein, their use is intended for distinguishing between similar elements and not necessarily for describing a sequential or chronological order. Moreover, relative terms like “front”, “back”, “top” and “bottom”, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as it is exemplary in nature, serving to describe the best mode of the invention known to the inventors as of the filing date hereof. Consequently, changes may be made in the arrangement and/or function of any of the elements described in the exemplary embodiments disclosed herein without departing from the spirit and scope of the invention.

This invention makes it possible to re-create the knee articulation as it was naturally, as it considers not only the whole knee motion behavior of the patient (current and pre-pathology), but also his individual HKA (Hip-Knee-Ankle) alignment. The articulation of the patella may also be taken into account.

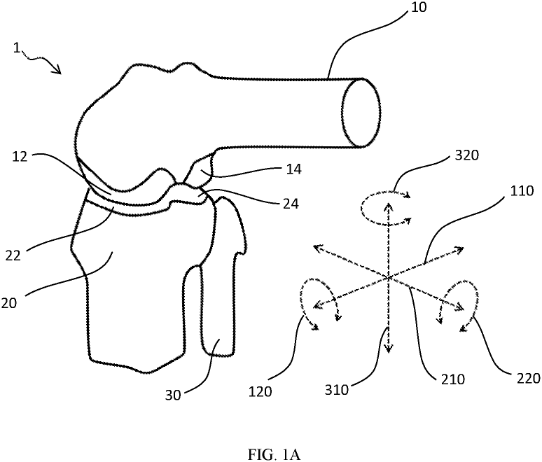

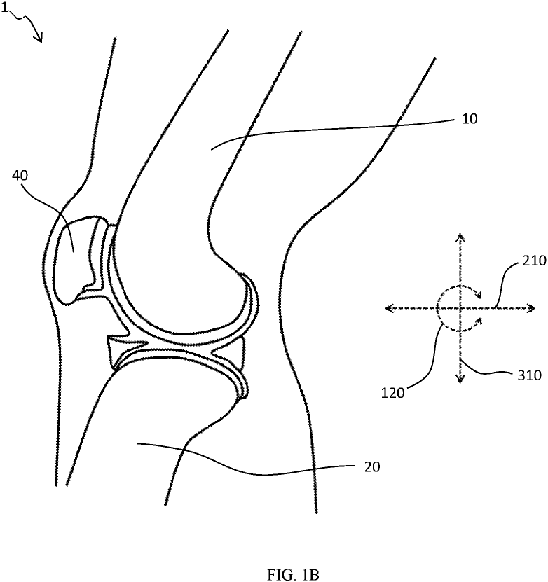

Referring to FIGS. 1A-1B, the knee articulation (1) in a patient’s sitting position (knee flexion) is shown in which the femur (10) and the tibia (20) are joined through the medial meniscus (22) and lateral meniscus (24), the contact on the femur being made on the medial condyle (12) and the lateral condyle (14). Applicants have identified that the natural relative movement of the femur vs tibia can be described as a sliding and rolling motion combination of linear movements such as medial/lateral translation (110), anterior/posterior translation (120), superior/inferior translation (310) and rotations such as flexion/extension (120), adduction/abduction (220), axial (internal/external) rotation (320). The knee movement can be characterized as movement according to a femoral helical axis, which is a combination of a flexion/extension axis and a longitudinal axis, and this resulting helical axis depends on the knee shape and knee alignment. This combined motion is different for each human being, and is modified by the medical condition of the patient (pathology, trauma, ...). The knee moves according to a femoral helical axis, which is a combination of a flexion / extension axis and a longitudinal axis, this resulting helical axis depends on the knee shape and knee alignment.

Well-known prior art for knee prostheses considers only the anterior/posterior translation (120) and the flexion/extension (120) rotation, which results in a rather simple prostheses, but has the drawback that the surgeon implants non-anatomically a non-anatomic implant. Such prostheses of the prior art have been found satisfying in terms of life duration (+93% after 15 years) but low in customer satisfaction (70%). Due to this situation, a number of patients who would benefit from a prosthesis delay the operation, fearing the risk of discomfort or pain. When discomfort and pain prevent the patient from sleeping quietly, then the patient asks for a knee prosthesis. The subject tries to wait as long as possible with medication, but if he waits too long, this may render the operation more difficult, as the knee deviation and pathology may increase and impact ligaments tension and collateral joints. In some countries, where the costs play a role, patients visit the doctor very late and so must do bilateral knee replacements at the same time (due to the damage caused the healthier knee by the prolonged load on the unhealthy knee).

A more recent generation of prostheses still uses the same limitation to the anterior/posterior translation (120) and the flexion/extension (120) rotation, but considers that an inclination of the flexion/extension (120) rotation can be made to obtain a movement that is closer to the natural motion of the patient’s knee. Since these prostheses are only in use for a few years, lifetime and patient satisfaction are not yet known. Nevertheless, even in this case, the surgeon attempts to anatomically implant a non-anatomic prosthesis.

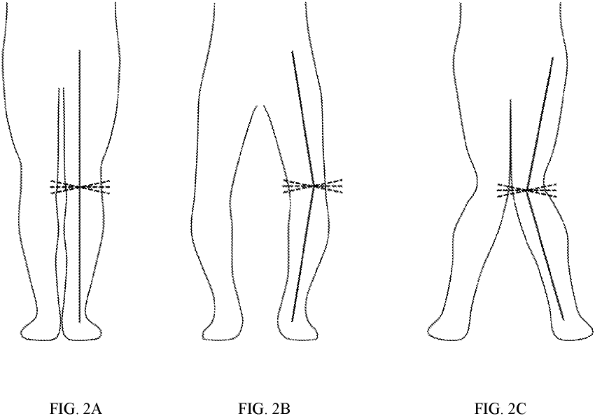

Referring now to FIGS. 2A-2B-2C, the invention described here considers a broader view of the patient’s constitutional anatomy. Human beings not only have a knee motion unique to the individual, characterized by an individualized knee joint morphology (patient-specific articulating surfaces between femur and tibia as well as between femur and patella), but they also have an individual HKA (Hip-Knee-Ankle) alignment. The normal (FIG. 2A) alignment, also called “neutral alignment” or “straight alignment”, is represented in a large part of the population, but signification variations can be observed, from constitutional varus (or bowleg) (FIG. 2B) to constitutional valgus (or knock knee) (FIG. 2C).

Current prior art considers that all patients should be brought to the normal alignment (FIG. 2A), and that an inclined installation of the prosthesis is sufficient to accommodate the patient’s individual knee articulation. Such alignment, also called « mechanical alignment », is characterized by neutral alignment and components positioned orthogonal to the femoral and tibial mechanical axes. The motion and attachment of the patella (40) are also neglected. Therefore. the knee prostheses of the prior art can be produced as a standard with only a few sizes, resulting in lower costs of production. The difficulty when using such prostheses of the prior art is to mechanically and anatomically position a non-anatomic prosthesis, which leads to many compromises in terms of positioning and sizing which sometimes results in prosthetic overhang (risk of pain, loss of mobility) or the implantation of an undersized prosthesis (which increases the risk of sinking, or loosening), so that the newly created knee articulation may generate new tensions, unwanted pressure in the ligaments holding the articulation together, and in the bones. The friction between these elements, which may generate discomfort or pain for the patient.

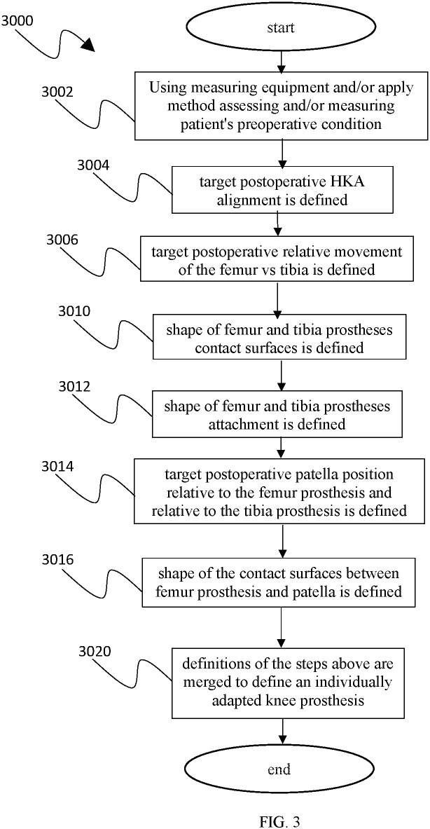

Referring now to FIG. 3, a method 3000 according to the invention consists of several steps, not necessarily in the following order. In a first step 3002, using CT-scan, X-ray, MRI, EOS (in loaded or unloaded conditions, monopodal, bipodal, under varus / valgus stress) or any other measuring equipment and/or apply any method known in the art, assessing and/or measuring patient’s preoperative condition, including assessing/measuring at least one of the following patient conditions:
(a) HKA alignment (See FIGS. 2A, 2B, 2C);
(b) relative movement of the femur vs. tibia (sliding and rolling motion combination as described in FIG. 1A);
(c) femur and tibia contact surfaces and bone shape; and
(d) patella shape and position relative to the femur and tibia, and contact surfaces of the patella with the femur.

In a second step 3004, target postoperative HKA alignment is defined according to the substep (a) above and to patient’s anatomic history (if known).

In a third step 3006, the target postoperative relative movement of the femur vs tibia (sliding and rolling motion combination) is defined according to the second step and substep (b) above.

In a fourth step 3010, the shape of femur and tibia prostheses contact surfaces is defined according to the second and third steps as well as substep (c) above.

In a fifth step 3012, the shape of femur and tibia prostheses attachment is defined according to the fourth step and substep (c) above.

In a sixth step 3014, the target postoperative patella position relative to the femur prosthesis and relative to the tibia prosthesis is defined according to the second, third and fourth steps as well as substep (d) above and to patient’s anatomic history (if known).

In a seventh step 3016, the shape of the contact surfaces between the femur prosthesis and the patella is defined according to the second, third, fourth and sixth steps as well as substep (d) above (contact surface only between the femoral component and the patella component, not between the patella component and the tibia component).

In an eighth step 3020, the above definitions are merged to define an individually adapted knee prosthesis.

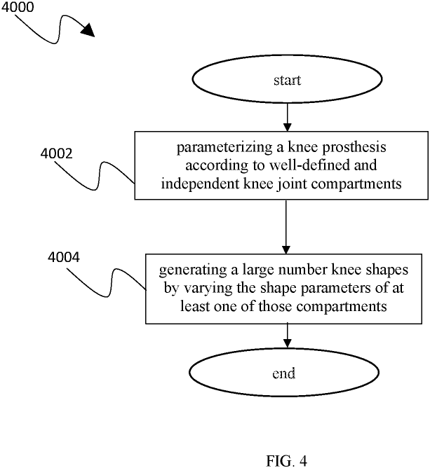

Referring now to FIG. 4, a method 4000 according to the invention consists of several steps, not necessarily in the following order. The method 4000 of selecting a 3D knee prosthesis model for a particular patient, the method consisting of:
Step 4002: (a) parameterizing a knee prosthesis according to well-defined and independent knee joint compartments, and
Step 4004: (b) generating a large number knee shapes by varying the shape parameters (surface and dimension) of at least one of those compartments,
wherein a suitable knee prosthesis may be selected from this generated knee shapes to match the patient’s needs as the generated configurations reproduce a high variability of knee shapes which reproduce the 3D shape asymmetries of each individual knee, thereby enabling the replication of the knee motion of the patient.Regarding step 4002 above, for every compartment, the parameterization is realised in such way that the two types of representations/sketches can be adjusted. The first type involves personalizing the articular representations/sketches defining the articular surface of each compartment by adapting the ML and AP guiding curves in each plane (radii of curvature, offset, position of the origin of those guiding curves to the patient’s knee frame) in order to match the 3D shape of the prosthetic surface with the patient’ knee surface. The second type involves personalizing the dimension sketches/representations (guiding curves defining the outside limits) along the joint surface in order to correctly fit the prosthetic dimension all around the joint to the patient’ size. Parameters define the orientation and position of those external guiding curves in each plane (radii, dimension, position of the origin of those guiding curves to the patient’ knee frame).

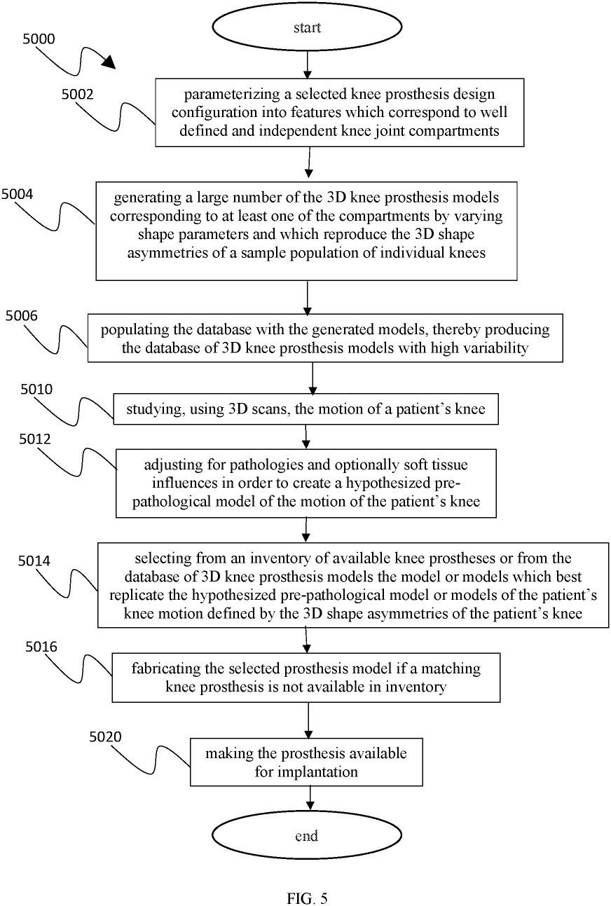

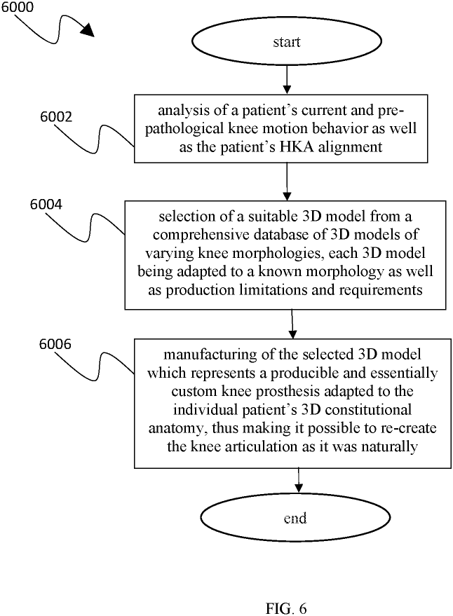

Referring now to FIG. 5, a method 5000 according to the invention consists of several steps, not necessarily in the following order. The method 5000 prepares a database of 3D knee prosthesis models from which can be selected a 3D knee prosthesis model that matches a particular patient’s needs. The method includes the following several steps, not necessarily in the following order.
Step 5002: (a) parameterizing a selected knee prosthesis design configuration into features which correspond to well defined and independent knee joint compartments;
Step 5004: (b) generating a large number of the 3D knee prosthesis models corresponding to at least one of the compartments by varying shape parameters such as surface and dimension and which reproduce the 3D shape asymmetries of a sample population of individual knees; and
Step 5006: (c) populating the database with the generated models, thereby producing the database of 3D knee prosthesis models with high variability;
Step 5010: (d) studying, using 3D scans, the motion of a patient’s knee;
Step 5012: (e) adjusting for pathologies and optionally soft tissue influences in order to create a hypothesized pre-pathological model of the motion of the patient’s knee;
Step 5014: (f) selecting from an inventory of available knee prostheses or from the database of 3D knee prosthesis models the model or models which best replicate the hypothesized pre-pathological model or models of the patient’s knee motion defined by the 3D shape asymmetries of the patient’s knee;
Step 5016: (g) fabricating the selected prosthesis model if a matching knee prosthesis is not available in inventory; and
Step 5020: (h) making the prosthesis available for implantation. Referring now to FIG. 6, a method 6000 according to the invention consists of several steps, not necessarily in the following order. A knee prosthesis is provided which is manufactured from a 3D model selected after applying the method 6000 including the steps of:
Step 6002: (a) analysis of a patient’s current and pre-pathological knee motion behavior as well as the patient’s HKA alignment,
Step 6004: (b) selection of a suitable 3D model from a comprehensive database of 3D models of varying knee morphologies, each 3D model being adapted to a known morphology as well as production limitations and requirements, and
Step 6006: (c) manufacturing of the selected 3D model which represents a producible and essentially custom knee prosthesis adapted to the individual patient’s 3D constitutional anatomy, thus making it possible to re-create the knee articulation as it was naturally.

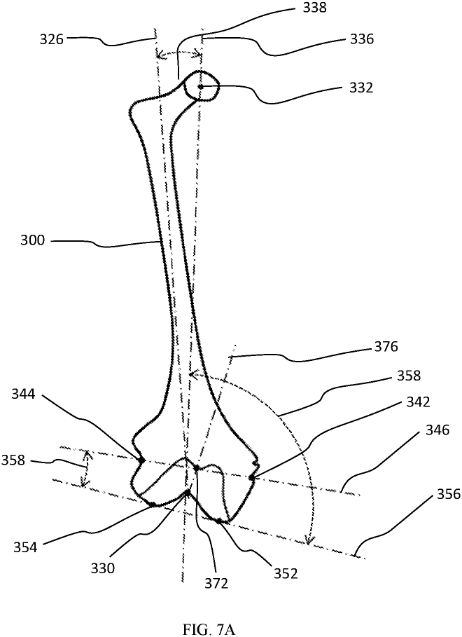

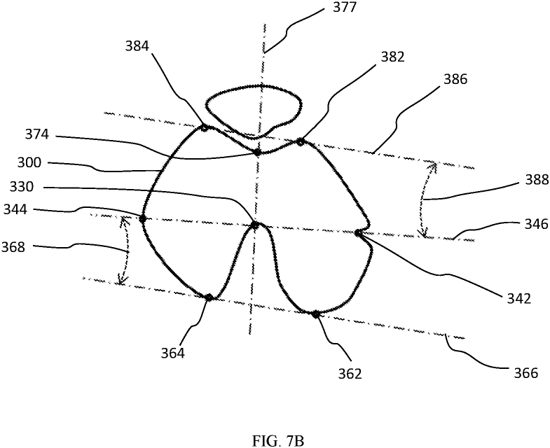

Referring now to FIGS. 7A and 7B, the femur is shown in an axial plane view, there is a very wide variation among the population with pre-arthritic or native knee (without any pathology on the knee joint), in terms of:
Knee dimensions
Knee shape
Knee size
Limb alignment.

Some individuals with healthy knee joints have either:
a constitutional varus alignment (O shape of the limb);
a constitutional valgus alignment (X shape of the limb); or
a constitutional neutral alignment (I shape of the limb).

The deviated limb alignment in those healthy knees (varus or valgus) is not considered as a misalignment, but simply as a deviated constitutional limb alignment.

Inside the knee joint, some parameters vary very widely, among the same population, thus for the same dimension of the knee, the shape itself may change significantly.

The main parameters, which act functionally on the joint are described below:
Alpha (α): Angle between FMA and BCD
DCA: Angle between TEA and BCD
PCA: Angle between TEA and BCP
ATA: Angle between TEA and TL
SA: Sulcus Axis (links KC to TG)
WL: Whiteside Line, links KC to TGL

All those parameters may vary with each other more than 15° (every angulated parameter can vary from its average value to 7.5° meaning that the range of variability of each parameter among the healthy population is estimated to 15° — a Gaussian curve).

Those variations may be due to genetics (hereditary), ontogenesis, posturology and / or activity and / or bodyweight during growth, before skeletal maturity, gender, morphotype (endomorphic, ectomorphic, mesomorphic) or ethnics (daily life activities linked to deep knee flexion).

There is for each knee, an intimate link between the geometry of the knee and the surrounding soft tissue envelope, especially with the cruciate, collateral and retinaculae ligaments. If the shape of the knee changes after implantation of a knee prosthesis, the relation between the insertion of the ligaments and the joint surface changes and this may not only create difficulty or even an inability to correctly balance the knee between the medial and lateral compartment, but also between the extension and the flexion as well as the midflexion.

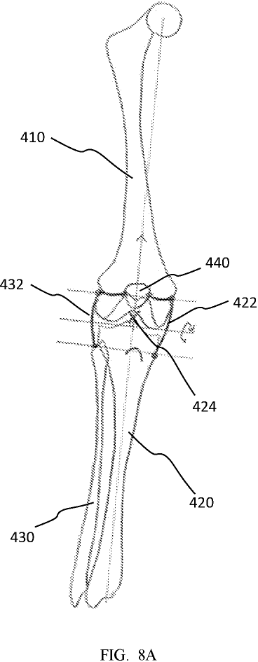

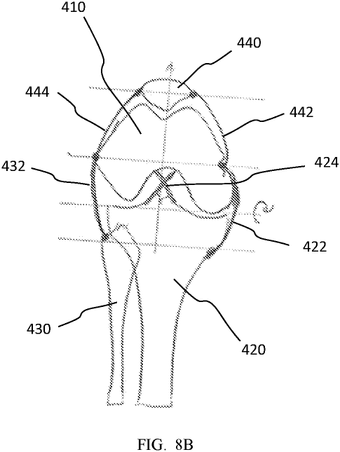

Referring now to FIGS. 8A to 8B, the intimate relation between knee alignment, knee size and shape, ligaments insertion and length (collateral ligaments, cruciate ligaments, retinaculae ligaments) are shown. The medial collateral ligament 422 attaches femur 410 and tibia 420. The cruciate ligaments 424 attach femur 410 and tibia 420. The lateral collateral ligament 432 attaches the femur 410 and fibula 430. Medial patellar retinaculum 442 attaches the femur 410 and patella 440 on the medial side. The lateral patellar retinaculum 444 attaches the femur and patella 440 on the lateral side.

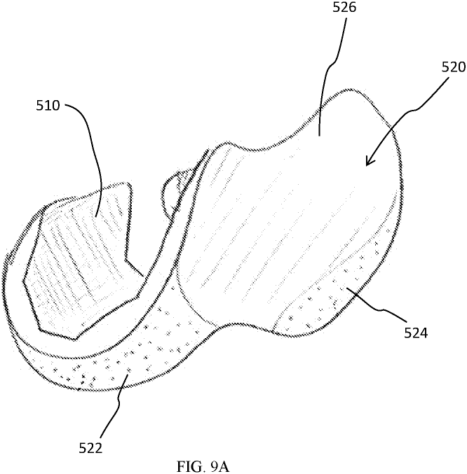

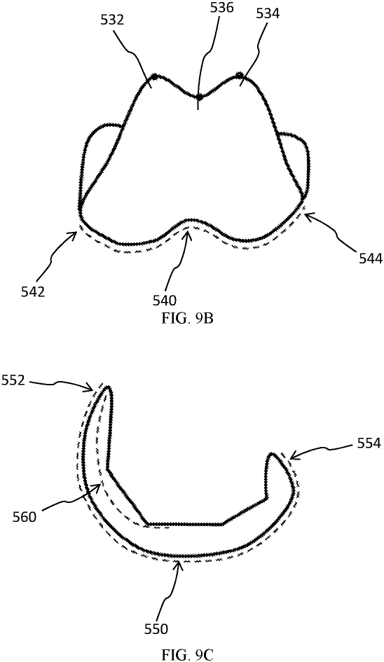

Referring now to FIGS. 9A to 9C, representing the femoral implant of a knee prosthesis of the invention, the following elements are shown for reference:
Bone facing surface 510,
Articular surface 520,
Condylar portion of the medial condyle 522,
Condylar portion of the lateral condyle 524,
Trochlear portion 526,
Medial elevation 532,
Lateral elevation 534,
Trochlear depth 536,
Medio-lateral curve 540, Medial end of the medio-lateral curve 542,
Lateral end of the medio-lateral curve 544,
J-curve condyles 550,
Anterior part of the J-curve condyles 552,
Posterior part J-curve condyles 554, and
J-curve trochlea 560.

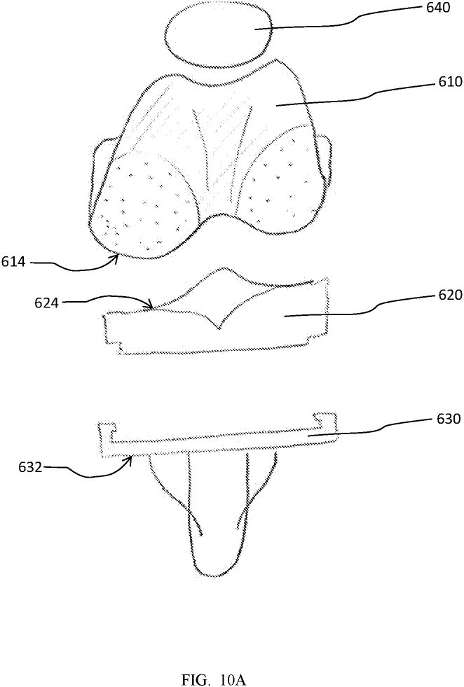

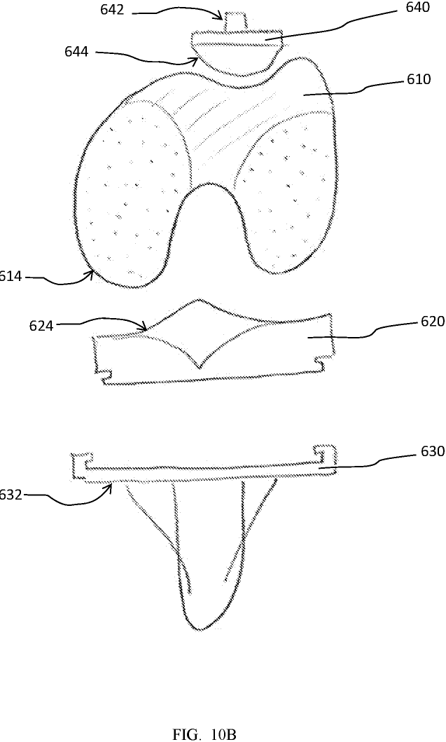

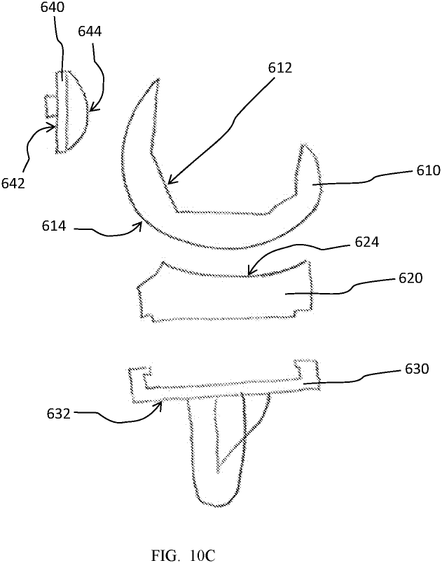

Referring now to FIGS. 10A to 10C, representing the knee prosthesis of the invention, the following elements are shown:
Femoral component 610,
Femoral inner bone-facing surface 612,
Outer articulating surface 614,
Tibial insert component 620,
Corresponding articulating surface 624,
Tibial tray component 630,
Tibial inner bone-facing surface 632,
Patella component 640,
Patella inner bone-facing surface 642, and
Patella articulating surface 644.

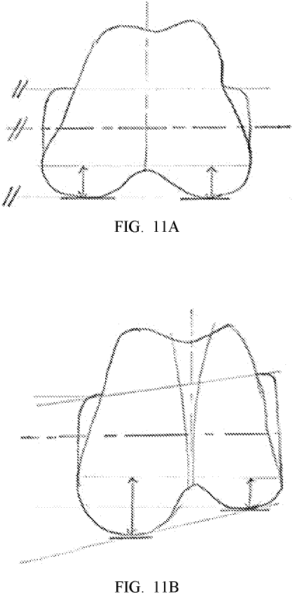

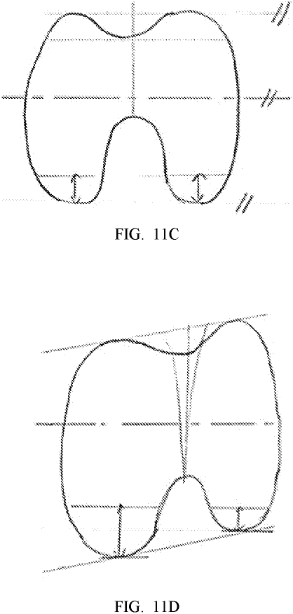

Referring now to FIGS. 11A to 11D, the comparison between a knee prosthesis of the prior art and the knee prosthesis according to the invention is made clearly visible.

### Comparison With Standard (Off-the-shelf) Knee Prostheses and Limits of This System

Because the knee prosthesis or knee implant is non-anatomic (in that it only roughly matches the anatomical movement of a human knee) and has to be implanted in a manner that more or less matches the anatomy of the patient’s knee joint, many significantly simplifications and compromises have to be integrated into the design of knee prostheses.

Initially the goal was to mechanically optimize the implantation of the prosthesis in order to obtain high longevity or life expectancy of the prosthesis. These knee prostheses have been designed to always be implanted mechanically (90° cuts to mechanical axis in the coronal plane), with both a symmetrical condyle shape and thickness and for a neutral limb realignment.

Then dogmatic concepts were implemented in the range of sizes of the knee prostheses of different companies in a compromise of the shape, size and alignment to an average knee and limb morphotype, which concepts include:
The number of sizes is typically limited to 10 sizes. However, this cannot realistically fulfil the wide variation in dimensions from small knees to very wide knees, because we know that the smallest sizes of the Caucasian population do not fit the corresponding sizes of the Asian population. Further, most companies increase sizes by adding a size in-between two sizes but not by increasing the extreme sizes.
The Medio-Lateral /Antero-Posterior ratio has been considered fixed, but we know now that it changes significantly among the population, and prosthetic overhang or component undersizing have been well described.
The sagittal shape of the condyles and / or trochlea (J-curve) has typically been simplified to a single or double or multiple radii, but we know now that some patients have a single or a multi-radii J-curve.
The femoro-patellar joint has always typically been simplified with a fixed oriented sulcus axis, but we know now that if the distal femur is in varus or in valgus, the sulcus axis is not oriented equally in such a way.
Fifth, the condylar offset is almost never considered except for Smith & Nephew’s Journey knee with a fixed offset of 2.5 mm for both the medial, the distal, and posterior condyles, but we know now, that the obliquity of the joint line changes significantly among the population and also between the distal and posterior condyles.

Today, clinical outcomes after 15 years of implantation demonstrate more than 93% of survivorship of the knee prosthesis. However, 30% of the population of patients complain of dissatisfaction after total knee replacement, either of pain, loss of mobility, or abnormal kinematics.

A further challenge today is also that the population requiring a total knee replacement is younger and still very active. Consequently, the function of the knee and global satisfaction is more and more important. The trend today is thus to do one’s best to install anatomically a non-anatomic knee prosthesis. The function will be improved but with a risk of a loss of longevity of the knee prosthesis, resulting in a higher risk of early component loosening.

### Knee Implantation (Standard Knee Implantation, Patient-Specific or Personalized/Individualized or Customized Implantation) is Described as Below

Referring now to FIGS. 9A to 9C and FIGS. 10A to 10C, a knee prosthesis includes a femoral component and a tibial tray component (with insert), and a patella component, and it is designed based on patient-specific data (from the literature, from cadavers, from 3D images) to define a standard range knee prosthesis or to define a patient-specific prosthesis.

An inner, bone-facing surface of the femoral component conforms to the corresponding surface of the femoral condyle. Alternatively, it can conform to one or more optimized bone cuts on the femoral condyle. However, the outer, articular surface of the component is enhanced to incorporate a smooth surface having a nearly constant radius in the coronal plane. The corresponding articular surface of the tibial tray (insert) has a surface contour in the coronal plane that is matched to the outer articular surface.

In certain embodiments, the articular surface of the component incorporates a sagittal curvature that positively-matches the patient’s existing or healthy sagittal radius.

Modern knee prosthesis:
Symmetrical condyles;
Same thickness between medial and lateral condyles for distal and posterior condyles, but can be different between distal and posterior;
Tangent of the distal and posterior condyles parallel to knee prosthesis flexion axes;
Elevation of the trochlea fixed and parallel to the tangent of the posterior condyle (axial plane);
No condylar offset (excepted for S&N, 2.5 mm offset);
Fixed sulcus axis oriented laterally at about 6° or eccentrically laterally;

The inventive activity of the invention also consists being able to vary the medial femoro-tibial joint to the lateral femoro-tibial joint to the femoro-patellar joint independently from each other. This can be defined as the variable parameters described below:

Personalized knee prosthesis:
Offset in the distal condyles (not a fixed value) = alpha distal angle ad
Offset in the posterior condyles (not a fixed value) = alpha posterior angle αp
Offset in the trochlear elevation (not a fixed value)
ad is equivalent or not to αp and is equivalent or not to the trochlear elevation angle (ATA)
DCA can be equivalent or not to αd
PCA can be equivalent or not to αp
SA is not a fixed value (sulcus axis = γ1)
WL is not a fixed value (Whiteside line = γ2)

### Difference Between Standard (STD), Off the Shelf (OTS) Prosthesis and the Personalized Knee Prosthesis of the Invention

Referring now to FIGS. 11A to 11D, a significant difference between a STD (OTS) prosthesis and the personalized knee prosthesis of the invention are shown. FIG. 11A (coronal plane) and 11C (axial plane) show a standard knee femoral component defined with the distal and posterior prosthetic joint lines (DCA, PCA), parallel to the prosthetic knee flexion axis, with both a fixed sulcus axis and Whiteside line orientation (SA, WL). The orientation of the trochlear line is also parallel or with a fixed angle in flexion (ATA).

Referring in particular to FIG. 11B (coronal plane) and 11D (axial plane), a personalized knee prosthesis is shown defined with the distal and posterior prosthetic joint lines inclined (or oblique) to the prosthetic knee flexion axis, whose distal and posterior angles (DCA, PCA) are independent from each other and having both an independent sulcus axis and Whiteside line orientation (SA, WL). The orientation of the trochlear line varies independently to the posterior condylar joint line (variable angle) in flexion (ATA).

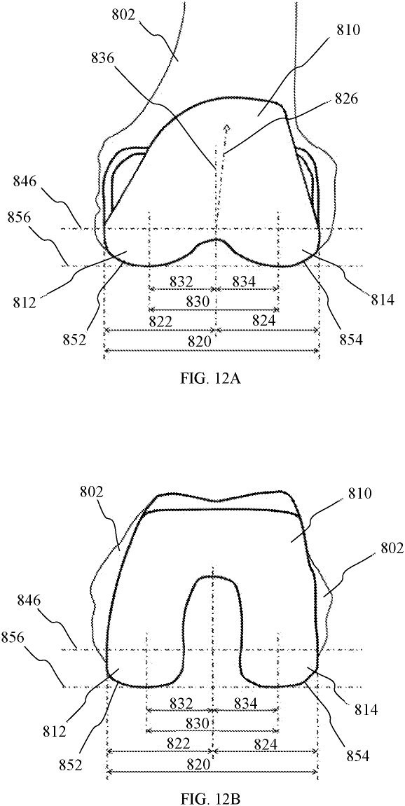

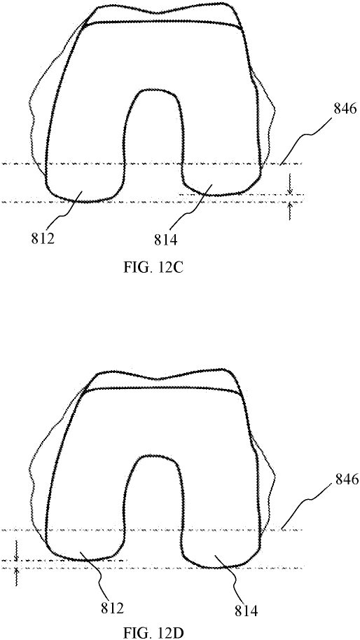

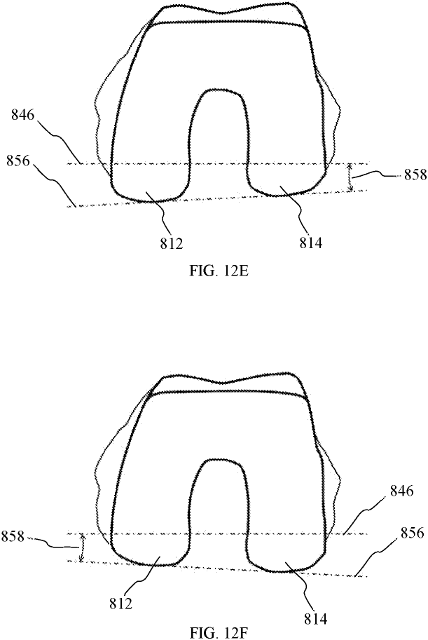

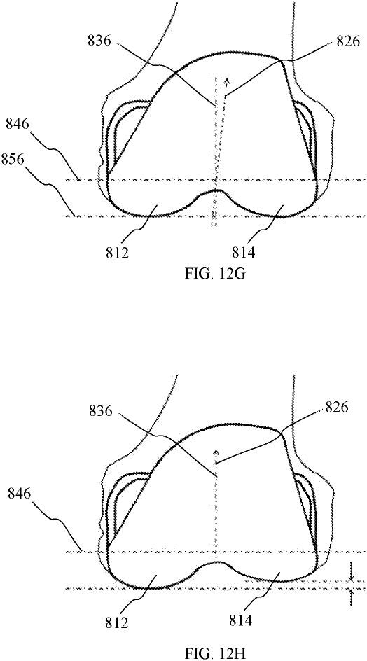

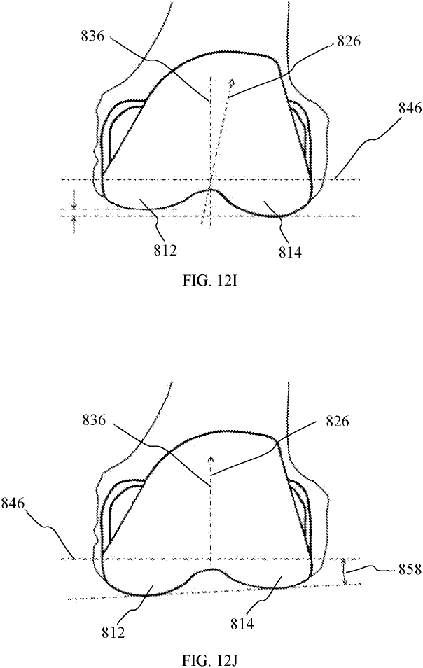

Referring now to FIGS. 12A-12K, the main geometrical parameters describing the femoral part of the knee prosthesis of the invention are defined as follows:
802: Femur
810: Femoral implant
812: Medial condyle
814: Lateral condyle
820: Mediolateral dimension (ML)
822: Medial condyle dimension from Femoral Mechanical Axis (FMA, 836)
824: Lateral condyle dimension from Femoral Mechanical Axis (FMA, 836)
826: Femoral Anatomical Axis (FAA)
830: Condyle interaxis
832: Medial condylar axis defined at the most distal point from Femoral Mechanical Axis (FMA, 836)
834: Lateral condylar axis defined at the most distal point from Femoral Mechanical Axis (FMA, 836)
836: Femoral Mechanical Axis (FMA)
838: HKS: Angle between FAA 826 and FMA 836
846: Transepicondylar Axis (TEA)
852: Medial condyle surface
854: Lateral condyle surface
856: Bicondyle Distal Tangent (BCD)
858: Angle between FMA 836 and BCD 856

Referring in particular to FIG. 12A and FIG. 12B, for a given mediolateral dimension (ML) 820, the medial condyle dimension 822 and the lateral condyle dimension 824 as measured from the femoral mechanical axis (FMA) 836 can be up to twice as wide as the other in the coronal (distal condyle) or axial (posterior condyles) plane.

Similarly, the medial condylar axis 832 and the lateral condylar axis 834 as measured from the femoral mechanical axis (FMA) 836 can vary from one another by up to 10 mm.

Referring to FIGS. 12C-12K,examples are shown of variations of the posterior and distal condyles made possible by a knee prosthesis of the invention.

Referring now to FIG. 12C, FIG. 12D, FIG. 12H, FIG. 12I, the size of the condyles 812 814 relative to each other can be made having a variation of up to 10 mm.

Referring now to FIG. 12E, FIG. 12F, FIG. 12J, FIG. 12K, the angle 858 between the femoral mechanical axis (FMA) 836 and the bicondyle distal tangent (BCD) 856 can vary up to 15° medially or laterally, distally or posteriorly.

Referring now to FIG. 12G, FIG. 12H, FIG. 12I, FIG. 12J, FIG. 12K, the variations and differences between the condyles 812, 814 can be adapted independently of the angle between the femoral anatomical axis (FAA) 826 and the femoral mechanical axis (FMA) 836, in other words all the adaptations of the condyles 812, 814 can be made independently from the prosthesis size and from the patient’s hip-knee-ankle (HKA) alignment, be it normal, varus or valgus.

Referring now to FIGS. 13A-13C, further parameters describing the femoral part of the knee prosthesis of the invention, in particular relating to the shapes, surfaces and contouring are defined as follows:
912: Medial condyle
914: Lateral condyle
936: Femoral Mechanical Axis (FMA)
946: Transepicondylar Axis (TEA)
952: Medial condyle surface
953: Trochlea surface
954: Lateral condyle surface
962: Shape radius of the medial condyle surface in the coronal and transverse plane
963: Shape radius of the trochlea surface in the coronal plane
964: Shape radius of the lateral condyle surface in the coronal and transverse plane
972: Shape of the medial condyle surface in the sagittal plane (medial condyle J-curve)
973: Shape of the trochlea surface in the sagittal plane (trochlea J-curve)
974: Shape of the lateral condyle surface in the sagittal plane (lateral condyle J-curve)
982: Medial contouring angle of the anterior face of the femoral implant in the coronal plane
984: Lateral contouring angle of the anterior face of the femoral implant in the coronal plane
992: Medial contouring angle of the posterior face of the femoral implant in the transverse and coronal plane
994: Lateral contouring angle of the posterior face of the femoral implant in the transverse and coronal plane

Referring now to FIG. 13A and FIG. 13B, the shape radius of the medial condyle surface in the coronal and transverse plane 962, the shape radius of the trochlea surface in the coronal plane 963 and the shape radius of the lateral condyle surface in the coronal and transverse plane 964 can be adjusted independently from each other and independently from the prosthesis size and from the patient’s hip-knee-ankle (HKA) alignment, be it normal, varus or valgus. It is important that the contouring (outside dimension of the implant) not overhang or uncover the resected area to resurface. The shape radius of the medial condyle surface in the coronal and transverse plane 962, the shape radius of the trochlea surface in the coronal plane 963 and the shape radius of the lateral condyle surface in the coronal and transverse plane 964 can vary from 15 mm to 65 mm. All contouring angles 982, 984, 992, 994 for the anterior and posterior faces of the femoral implant can be adjusted independently from each other and independently from the prosthesis size and from the patient’s hip-knee-ankle (HKA) alignment, be it normal, varus or valgus. All contouring angles 982, 984, 992, 994 for the anterior and posterior faces of the femoral implant can vary from 0° to 50°.

Referring now to FIG. 13C, the shape of the medial condyle surface in the sagittal plane (medial condyle J-curve) 972, the shape of the trochlea surface in the sagittal plane (trochlea J-curve) 973 and the shape of the lateral condyle surface in the sagittal plane (lateral condyle J-curve) 974 can be adjusted independently from each other and independently from the prosthesis size and from the patient’s hip-knee-ankle (HKA) alignment, be it normal, varus or valgus. The shape of the medial condyle surface in the sagittal plane (medial condyle J-curve) 972, the shape of the trochlea surface in the sagittal plane (trochlea J-curve) 973 and the shape of the lateral condyle surface in the sagittal plane (lateral condyle J-curve) 974 can be made of one radius but can also be made of a combination of two or more radii, which dimension can vary from 15 mm to 65 mm. When using a combination of radii, the transition from one radius to the next radius can be smoothened by the use of a spline or any other appropriate curve via, for example, application of curve or surface fitting algorithm such as offered by “SOLIDWORKS”™.

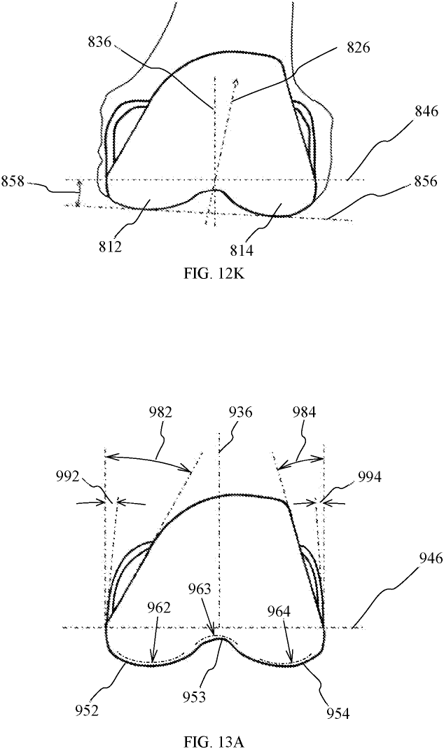

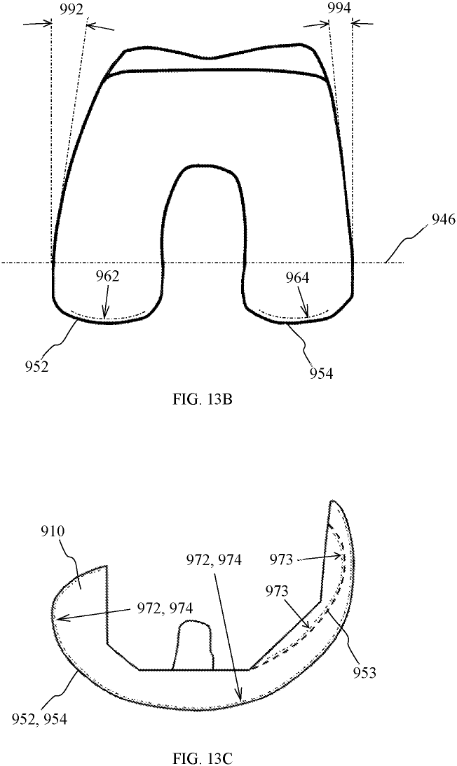

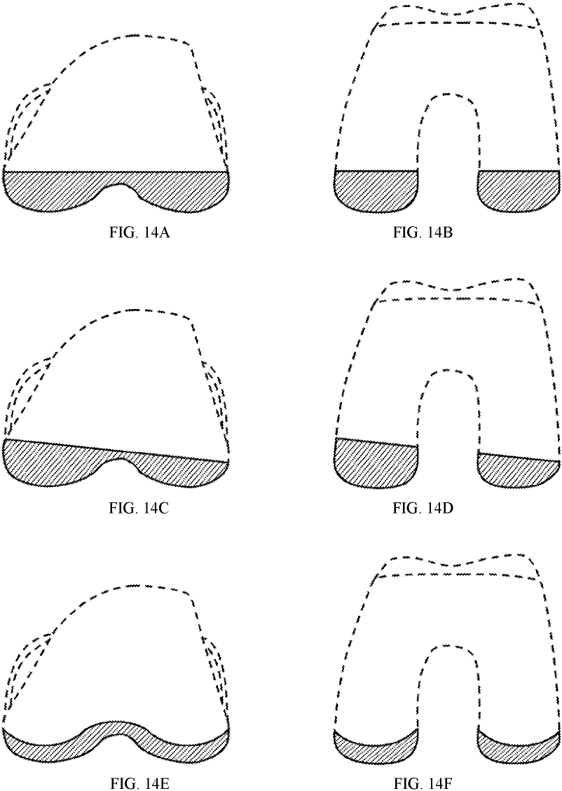

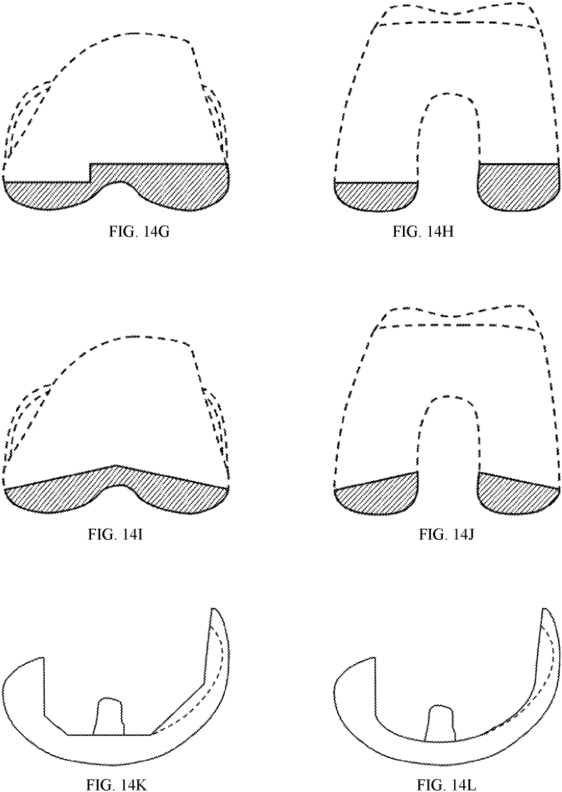

Referring now to FIGS. 14A-14L, the femoral part of the knee prosthesis of the invention can be adapted to any bony resections, such as plane (FIG. 14A, FIG. 14B), an incline (FIG. 14C, FIG. 14D), curve (FIG. 14E, FIG. 14F), offset (FIG. 14G, FIG. 14H), double-incline (FIG. 14I, FIG. 14J), or any other bony resection adapted to the individual needs of the patient. The bone resection can be sawed (FIG. 14K) or milled (FIG. 14L), or any other appropriate technique may be used as known in the industry.

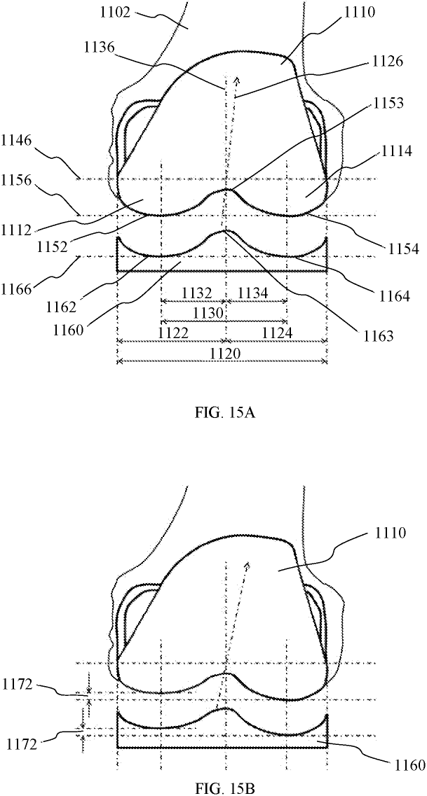

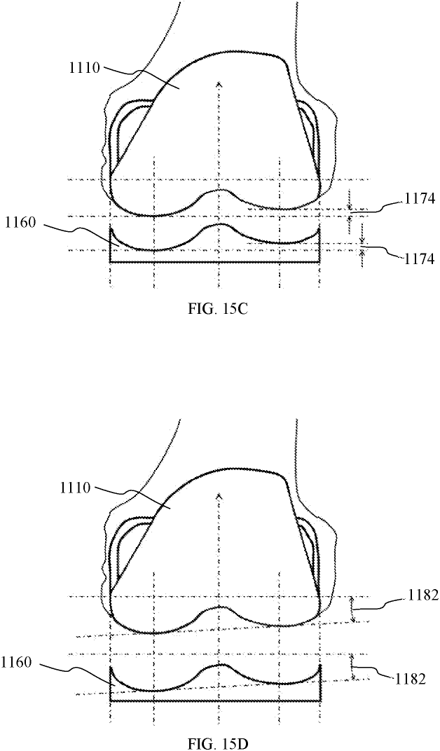

Referring now to FIGS. 15A to 15E, the main geometrical parameters describing the femoral implant and the tibial insert component of a knee prosthesis of the invention are defined as follows:
1102: Femur
1110: Femoral implant
1112: Medial condyle
1114: Lateral condyle
1120: Mediolateral dimension (ML)
1122: Medial condyle dimension from Femoral Mechanical Axis (FMA, 1136)
1124: Lateral condyle dimension from Femoral Mechanical Axis (FMA, 1136)
1126: Femoral Anatomical Axis (FAA)
1130: Condyle interaxis
1132: Medial condyle central axis from Femoral Mechanical Axis (FMA, 1136)
1134: Lateral condyle central axis from Femoral Mechanical Axis (FMA, 1136)
1136: Femoral Mechanical Axis (FMA)
1146: Transepicondylar Axis (TEA)
1152: Medial condyle surface of the femoral implant
1153: Trochlea surface of the femoral implant
1154: Lateral condyle surface of the femoral implant
1156: Bicondyle Distal Tangent (BCD) of the femoral implant
1160: Tibial insert component
1162: Medial condyle corresponding surface of the tibial insert component
1163: Trochlea corresponding surface of the tibial insert component
1164: Lateral condyle corresponding surface of the tibial insert component
1166: Bicondyle distal tangent of the tibial insert component

In a knee prosthesis according to the invention, the tibial insert component 1160 is made to match (“match” meaning for example via the application of known curve/surface fitting and smoothing techniques between shapes as mentioned, e.g., using “SOLIDWORKS”™ or the like, that interact across adjacent bone compartments) the femoral implant 1110, in other words the tibial insert component 1160 shares with the femoral implant 1110:
the same or essentially the same mediolateral dimension 1120,
the same or essentially the same medial condyle dimension from femoral mechanical axis 1122,
the same or essentially the same lateral condyle dimension from femoral mechanical axis 1124,
the same or essentially the same condyle interaxis 1130,
the same or essentially the same medial condyle central axis from femoral mechanical axis 1132,
the same or essentially the same lateral condyle central axis from femoral mechanical axis 1134,
the same or essentially the same transepicondylar axis 1146, such that:
   the medial condyle surface of the femoral implant 1152 fits to the medial condyle corresponding surface of the tibial insert component 1162,
   the trochlea surface of the femoral implant 1153 fits to the trochlea corresponding surface of the tibial insert component 1163,
   the lateral condyle surface of the femoral implant 1154 fits to the lateral condyle corresponding surface of the tibial insert component 1164, and
   the bicondyle distal tangent of the tibial insert component 1166 fits to the bicondyle distal tangent of the tibial insert component 1166.

Referring now to FIGS. 15B to 15E, a few examples of adaptations of the knee prosthesis of the invention to the need of an individual patient are shown.

Referring in particular to FIG. 15B, showing a knee prosthesis of the invention in the case of constitutional femur varus where the femoral implant 1110 has the distal lateral condyle shorter than the distal medial condyle, generating an offset 1172 between the condyles, essentially the same offset 1172 is reproduced in the tibial insert component 1160.

Referring now to FIG. 15C, showing a knee prosthesis of the invention in the case of constitutional femur valgus where the femoral implant 1110 has the distal lateral condyle longer than the distal medial condyle generating an offset 1174 between the condyles, the same or essentially the same offset 1174 is reproduced in the tibial insert component 1160.

Referring now to FIG. 15D, showing a knee prosthesis of the invention in the case of constitutional femur varus where the femoral implant 1110 has an angle 1182 opening from the distal lateral condyle to the distal medial condyle, the same or essentially the same angle 1182 is reproduced in the tibial insert component 1160.

Referring now to FIG. 15E, showing a knee prosthesis of the invention in the case of constitutional femur valgus where the femoral implant 1110 has an angle 1184 opening from the distal medial condyle to the distal lateral condyle, the same or essentially the same angle 1184 is reproduced in the tibial insert component 1160.

In a knee prosthesis of the invention, the offsets 1172, 1174 can vary from 0 to 10 mm, and the angles 1182, 1184 can vary from 0° to 15°.

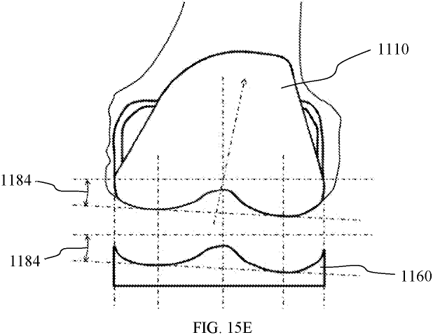

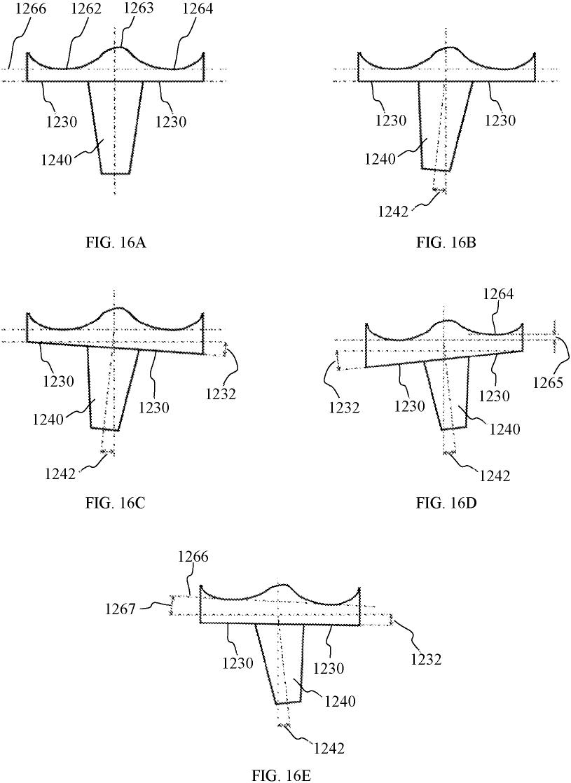

Referring now to FIGS. 16A-16E, showing schematically coronal views combining the geometries of the tibial insert, the tibial tray and the keel of a knee prosthesis of the invention, the main parameters in the coronal plane are defined as follows:
1262: Medial condyle corresponding surface of the tibial insert component
1263: Trochlea corresponding surface of the tibial insert component
1264: Lateral condyle corresponding surface of the tibial insert component
1266: Bicondyle distal tangent of the tibial insert component

In practice, the tibial insert, the tibial tray and the keel can be realized in one or more parts, and assembled by any of the appropriate techniques as well known in the industry, such assembly may be done before or during the surgery. For the purpose of the present explanation, these elements are presented as if made in one part with different sections: (i) the articulation surface of the tibial insert comprising the medial condyle corresponding surface 1262, the trochlea corresponding surface 1263 and the lateral condyle corresponding surface 1263, (ii) the bone facing surface of the tibial tray 1230 and (iii) the keel 1240. In a knee prosthesis of the invention, any orientation angle, any offset and any combination thereof applied to the tibial insert, the tibial tray and the keel so as to adapt to the need of the individual patient.

Referring now to FIG. 16B, the keel 1240 is not orthogonal to the bone facing surface 1230, but is oriented at an angle 1242 so as to adapt to the need of the individual patient.

Referring now to FIG. 16C, the keel 1240 is not orthogonal to the bone facing surface 1230, but is oriented at an angle 1242, and the bone facing surface 1230 is oriented at an angle 1232 so as to adapt to the need of the individual patient. As in this example, a thinner medial insert thickness favours the knee to be orientated in varus.

Referring now to FIG. 16D, the keel 1240 is not orthogonal to the bone facing surface 1230, but is oriented at an angle 1242, the bone facing surface 1230 is oriented at an angle 1232, and the lateral condyle corresponding surface 1264 presents an offset 1265, so as to adapt to the need of the individual patient.

Referring now to FIG. 16E, the keel 1240 is not orthogonal to the bone facing surface 1230, but is oriented at an angle 1242, the bone facing surface 1230 is oriented at an angle 1232, and the bicondyle distal tangent 1266 is oriented at an angle 1267, so as to adapt to the need of the individual patient. In a knee prosthesis of the invention, the offset 1265 can vary from 0 to 10 mm, and the orientation angles 1232, 1242, 1267 can vary up to 12°.

Typically, the offset can vary from -10° to +10° in mediolateral or in anteroposterior dimensions, and can be oriented up to 12° around the longitudinal axis of the keel.

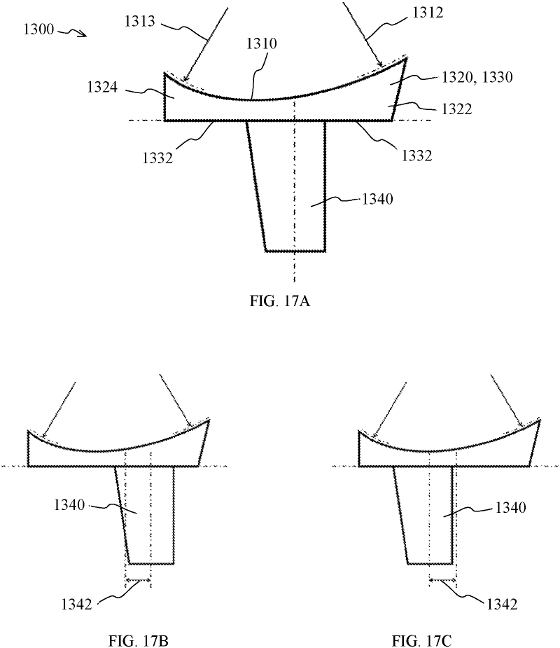

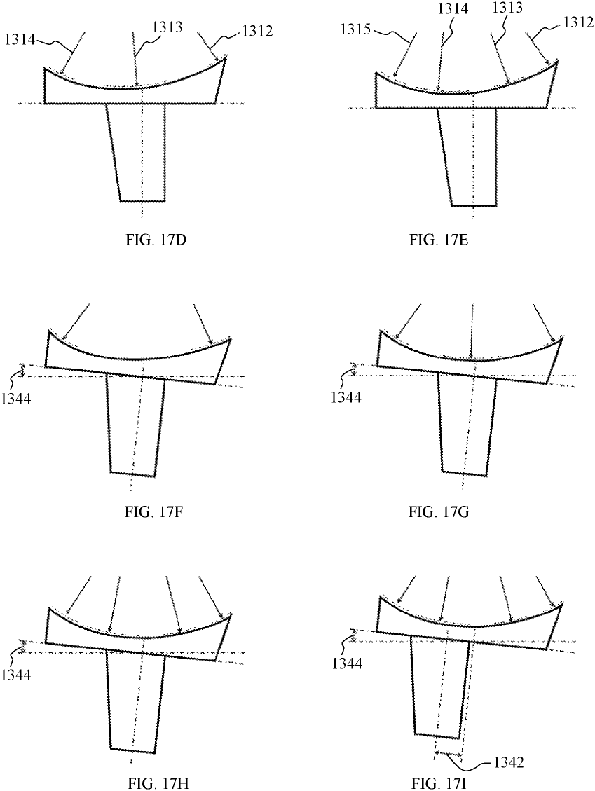

Referring now to FIGS. 17A-17I, showing schematically sagittal views combining the geometries of the tibial insert 1320, the tibial tray 1330 and the keel 1340 of a knee prosthesis of the invention, as if they were made in one piece 1300, in practice the tibial component 1300 of a knee prosthesis of the invention can be realized in one or more parts, and assembled by any of the appropriate techniques as well known in the industry, such assembly may be done before or during the surgery. On these simplified figures only one sagittal J-curve 1310 is represented, however this J-curve 1310 can equally represent the surfaces of the tibial component 1300 on which the medial condyle, the trochlea or the lateral condyle of the femoral implant of the invention interfaces.

The sagittal J-curve 1310 is adapted to fit with the corresponding surface of the femoral implant of the invention, so that the functioning of the knee prosthesis of the invention fits the need of the individual patient. To this end, the sagittal J-curve 1310 can be made of one radius but can also be made of a combination of two radii 1312, 1313 (FIG. 17A, FIG. 17B, FIG. 17C, FIG. 17F), three radii 1312, 1313, 1314 (FIG. 17D, FIG. 17G) or more radii 1312, 1313, 1314, 1315 (FIG. 17E, FIG. 17H, FIG. 17I), which dimension can vary from 15 mm to 80 mm.

To fit with the tibia of the individual patient, in the sagittal plane the keel 1340 of the tibial component 1300 of a knee prosthesis of the invention can be placed at the center of the tibial component 1300 (FIG. 17A, FIG. 17D, FIG. 17E, FIG. 17F, FIG. 17G, FIG. 17H), can present an offset 1342 towards the anterior part of the tibia (FIG. 17B) or towards the posterior part of the tibia (FIG. 17C, FIG. 17I). In a knee prosthesis of the invention, the offset 1342 can vary from 0 to 10 mm.

For the same purpose of fitting to the tibia of the individual patient, in the sagittal plane the bone facing surface 1332 of the tibial component 1300 of a knee prosthesis of the invention can be oriented at an angle 1344 (FIG. 17F, FIG. 17G, FIG. 17H, FIG. 17I). In a knee prosthesis of the invention, the angle 1344 can vary up to 12°.

The tibial component 1300 of a knee prosthesis of the invention can combine any and all the characteristics described in the present disclosure so as to better match the needs of the individual patient (FIG. 17I).

Typically, the offset can vary from -10° to +10° in mediolateral or in anteroposterior dimensions, and can be oriented up to 12° around the longitudinal axis of the keel.

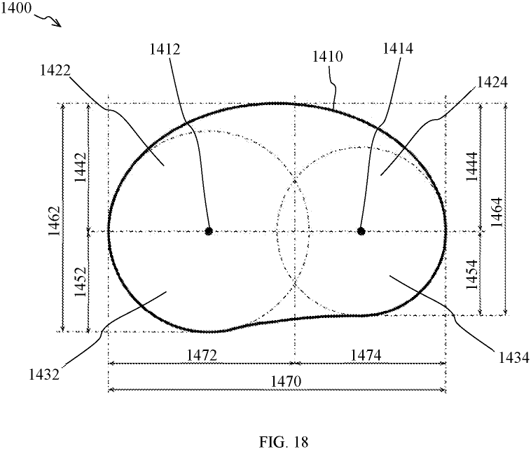

Referring now to FIG. 18, the parameters defining the contouring 1410 of the tibial component 1400 are as follows:
1412: Medial condyle
1414: Lateral condyle
1422: Anterior medial condyle
1424: Anterior lateral condyle
1432: Posterior medial condyle
1434: Posterior lateral condyle
1442: Anteroposterior dimension of the anterior medial condyle (APam)
1444: Anteroposterior dimension of the anterior lateral condyle (APal)
1452: Anteroposterior dimension of the posterior medial condyle (APpm)
1454: Anteroposterior dimension of the anterior lateral condyle (APal)
1462: Anteroposterior dimension of the medial condyle (APm)
1464: Anteroposterior dimension of the lateral condyle (API)
1470: Mediolateral dimension (ML)
1472: Mediolateral dimension of the medial condyle (MLm)
1474: Mediolateral dimension of the lateral condyle (MLl)

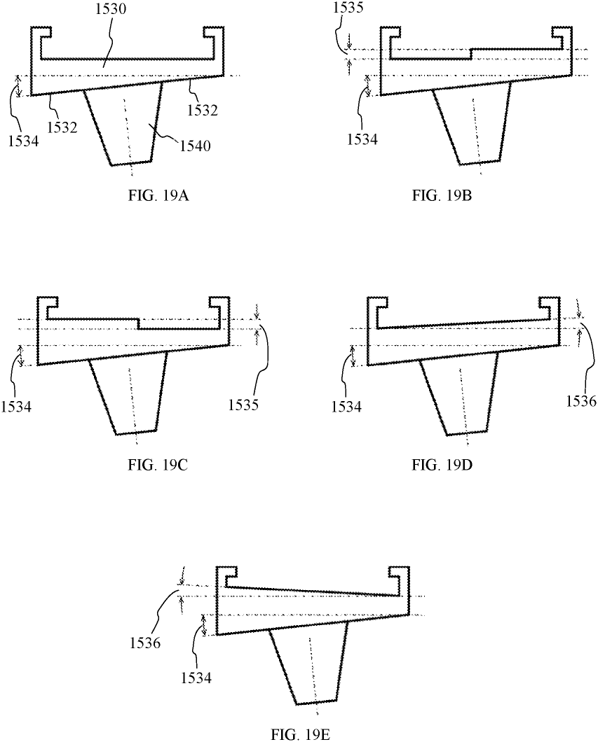

In a knee prosthesis of the invention, all these parameters can be adapted to fit with the needs of the individual patient. In such prosthesis, the mediolateral dimension 1470 can vary from 40 mm to 150 mm, the mediolateral dimensions of the condyles 1472, 1474 can both and independently vary from 15 mm to 70 mm, and the anteroposterior dimensions of the condyles 1442, 1444 can both and independently vary from 30 mm to 70 mm. Referring now to FIGS. 19A-19E, the tibial tray 1530 of a knee prosthesis of the invention can be adapted to fit to the needs (optimized component coverage and anchorage) of the individual patient.

In the coronal plane, the bone facing surface 1532 can present a certain orientation angle 1534, and the surface where the tibial insert is attached can present a certain offset 1535 (FIG. 19B, FIG. 19C) or another orientation angle 1536 (FIG. 19D, FIG. 19E). In a knee prosthesis of the invention, the angles 1534, 1536 can vary from -12° to +12°, and the offset 1535 can vary from 0 to 10 mm in medial and/or lateral dimensions and be oriented up to 45°. The tibial tray 1530 of a knee prosthesis of the invention can combine any and all the characteristics described in the present disclosure so as to match the needs of the individual patient.

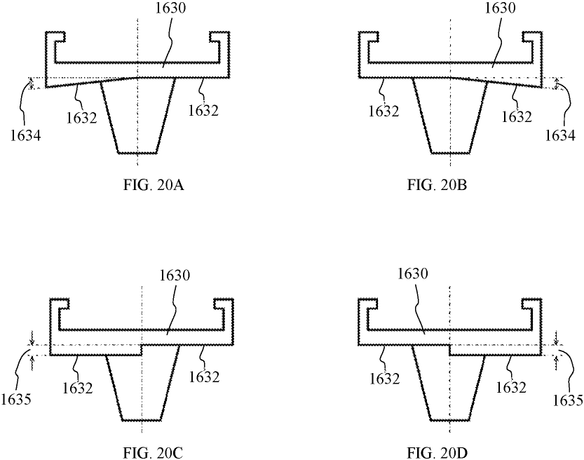

Referring now to FIGS. 20A-20D, the tibial tray 1630 of a knee prosthesis of the invention can be adapted to any bone resection geometry to fit to the needs of the individual patient. In the coronal plane, the bone facing surface 1632 can be made with a certain resection angle 1634 on the medial side (FIG. 20A) or on the lateral side (FIG. 20B), or a resection step 1635 on the medial side (FIG. 20C) or on the lateral side (FIG. 20D). In a knee prosthesis of the invention, the angle 1634 can vary from -12° to +12°, and the step 1635 can vary from 0 to 10 mm in medial and/or lateral and be oriented up to 45°. The tibial tray 1630 of a knee prosthesis of the invention can combine any and all the characteristics described in the present disclosure so as to fit with the need of the individual patient.

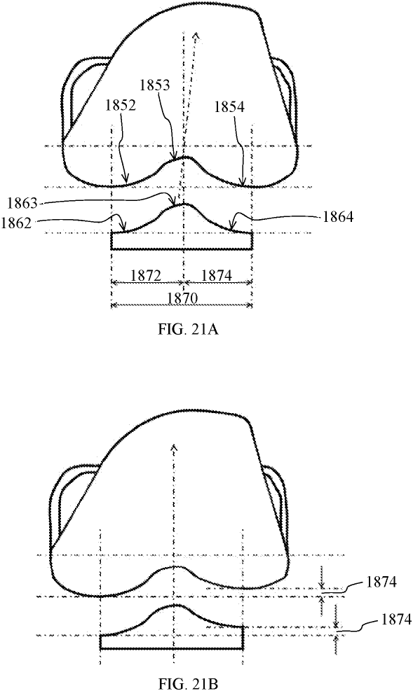

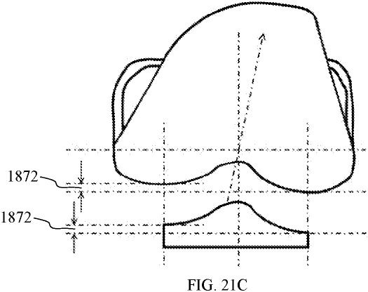

Referring now to FIGS. 21A to 21C, in the coronal plane, the parameters defining the geometry of the patella component of a knee prosthesis of the invention are as follows:
1852: Medial condyle surface of the femoral implant
1853: Trochlea surface of the femoral implant
1854: Lateral condyle surface of the femoral implant
1862: Articulating surface of the patella component corresponding to the medial condyle
1863: Articulating surface of the patella component corresponding to the trochlea
1864: Articulating surface of the patella component corresponding to the lateral condyle
1870: Mediolateral dimension of the patella component
1872: Mediolateral dimension of the medial side of the patella component
1874: Mediolateral dimension of the lateral side of the patella component

The geometry of the patella component of a knee prosthesis of the invention is adapted to fit the need of the individual patient. The articulating surfaces of the patella component 1862, 1863, 1864 are made to fit to their respective corresponding surfaces of the femoral component 1852, 1853, 1854, also taking into account the patient’s hip-knee-ankle (HKA) alignment, be it normal, varus or valgus. As an example, in the case the femoral component has an offset (1872 in FIG. 21C, 1874 in FIG. 21B) between the distal lateral condyle and the distal medial condyle, the offset 1872, 1874 is reproduced in the patella component.

Referring now to FIGS. 22A and 22B, showing the patella component of a knee prosthesis of the invention, the mediolateral surfaces (FIG. 22A) can be symmetrical or asymmetrical between medial and lateral compartment, and the anteroposterior surfaces (FIG. 19B) can be symmetrical or asymmetrical between the anterior and posterior compartment so as to match the needs of the individual patient. FIG. 22A is a side view showing medial and lateral compartment. FIG. 22B is a side view showing anterior and posterior compartment. The four compartments may or may not be symmetrical.

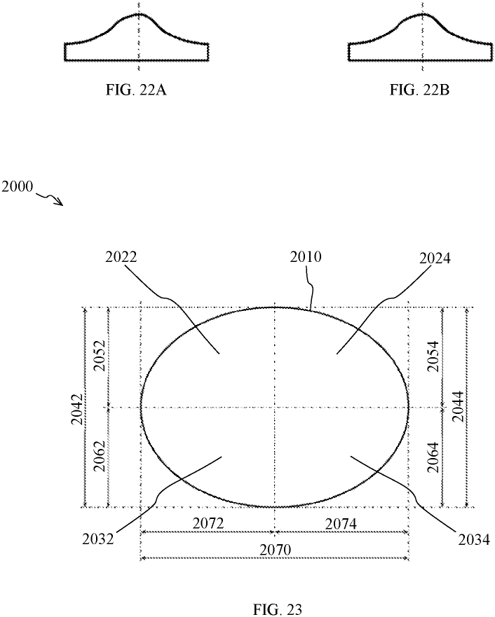

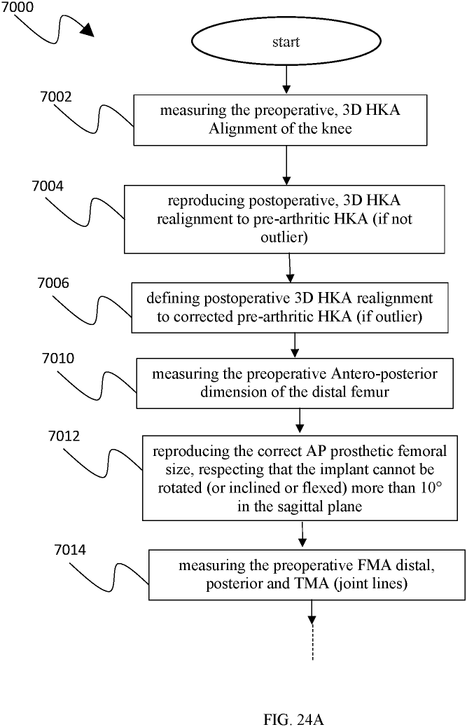

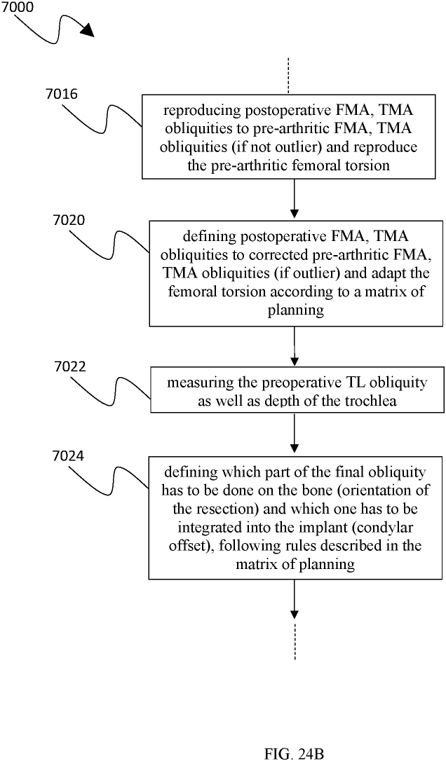

Referring now to FIG. 23, the parameters defining the contouring 2010 of the patella component 2000 are as follows:
2022: Anterior medial compartment
2024: Anterior lateral compartment
2032: Posterior medial compartment
2034: Posterior lateral compartment
2042: Anteroposterior dimension on the medial side (APm)
2044: Anteroposterior dimension on the lateral side (API)
2052: Anteroposterior dimension of the anterior medial compartment (Appam)
2054: Anteroposterior dimension of the anterior lateral compartment (APal)
2062: Anteroposterior dimension of the posterior medial compartment (APpm)
2064: Anteroposterior dimension of the posterior lateral compartment (APpl)
2070: Mediolateral dimension (ML)
2072: Mediolateral dimension of the medial side (MLm)
2074: Mediolateral dimension of the lateral side (MLl)

In a knee prosthesis of the invention, all these parameters can be adapted to fit with the needs of the individual patient. Each of the 4 compartments (medial, lateral, anterior, posterior) can be specified to fall in the range from 8 mm to 30 mm in width and height. The values are independent of the patella thickness, which should be at least 6 mm or more.

### Main Features Around the Invention

The invention’s design specificities for the distal and posterior condyles to the trochlea part of the femoral component are personalizable features that, when put together, realign the pathologic limb as it was before the pathology and which reshape the joint surface as close as possible to the pre-arthritic shape and size of the knee joint.

The different types of correction:
from the pathologic deviated limb alignment to the pre-arthritic limb alignment;
from the pathologic knee joint surface to the pre-arthritic knee joint surface (the two condyles and the trochlea), which implicates the medio-lateral curves of the articular surface as well as the sagittal J-curves of both condyles and trochlea;
from the pathologic femoral (distal, posterior and in-between), tibial proximal & trochlear joint lines to the corresponding pre-arthritic joint lines (=condylar offset, trochlear offset between medial and lateral compartments);
from the pathologic distance between the depth of the trochlea to the medial and lateral trochlear elevation to the corresponding pre-arthritic distance;
from the pathologic coronal distance between the sagittal axis of the condyles and the trochlea to the middle of the knee to the corresponding pre-arthritic distance;

Supplementary corrections (or rules that we internally use to avoid outliers in terms of alignment and shapes and whose range defines what is normality and abnormality for alignment and shape and thus indicate what is replicated or adjusted) from the native or pre-arthritic limb alignment and knee shape may be made:
when the pre-arthritic limb alignment is considered as mechanically unstable (too deviated, if HKA postop more than 5° in varus or in valgus HKA <175° or HKA >185°, risk of loosening of the components because of an imbalance in load sharing because of excessive abduction or adduction moment), a correction of the limb alignment is applied to restrict the global alignment between 175° < HKA postop < 185°;
when the pre-arthritic knee joint lines are considered as mechanically not stable (too oblique condyles, if FMA postop more than 5° FMA < 85° or FMA > 95°, TMA < 85° or TMA > 95°, risk of loosening of the components because of imbalance load sharing), a correction of the condylar offset is applied to restrict the obliquity of the condyles and proximal tibia to 5° (85° < FMA, TMA < 95°). This also happens when the medial or lateral condyle is under-developed (hypoplasia) or when the bone wear in a condyle is excessive;
when the pre-arthritic limb alignment has to be corrected, this means that a restricted or hybrid alignment is targeted and the postop limb alignment is an in-between alignment between the anatomical deviated pre-arthritic and a neutral postop alignment (in this condition, a compromise is made with the FMA (femoral distal joint line obliquity). This compromise has to be moved back/reported posteriorly to the condyles to balance the correction during the whole flexion).
when the pre-arthritic lateral elevation of the trochlea is too flat, this may lead to an unstable patella during flexion (patellar dislocation), thus a lateral wall will be built (at least 5 mm) to secure and correctly center the patella during the flexion;
when the pre-arthritic tibia slope is too excessive or insufficient (excessive downslope or upslope), this may lead to a loss of mobility or contribute to an excess of extension (recurvatum) with associated knee joint laxity, and therefore a correction is systematically applied such that the posterior tibia slope falls within the range of 0° < TPS < 10°.

### Method of Making the Personalized Prosthesis

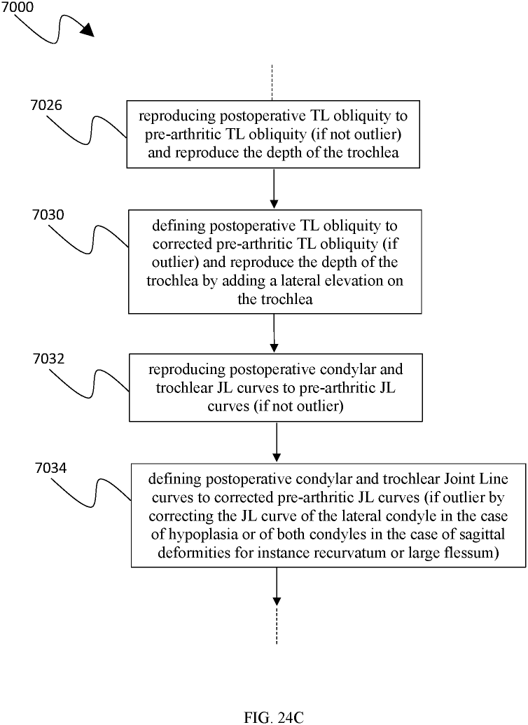

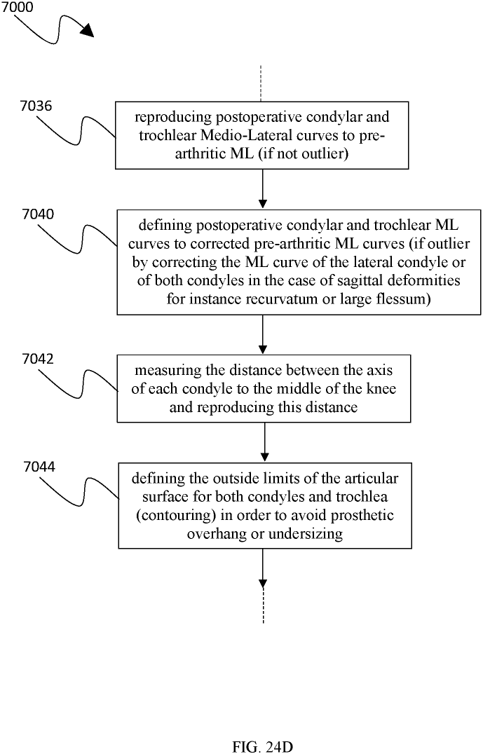

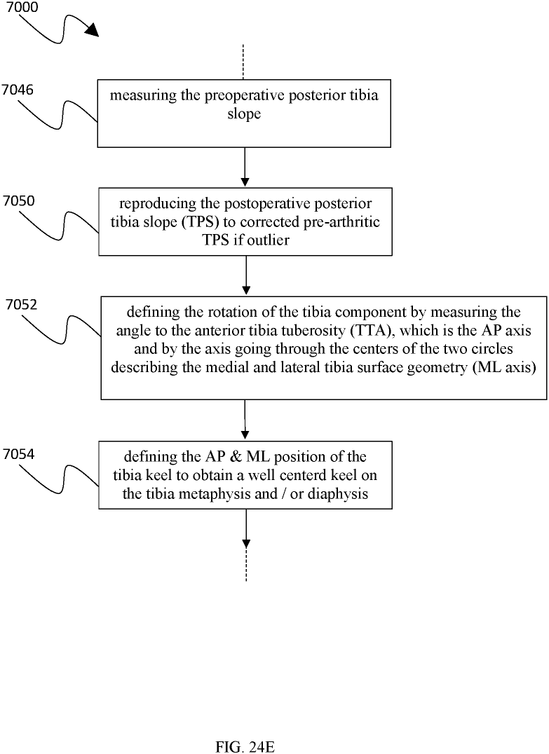

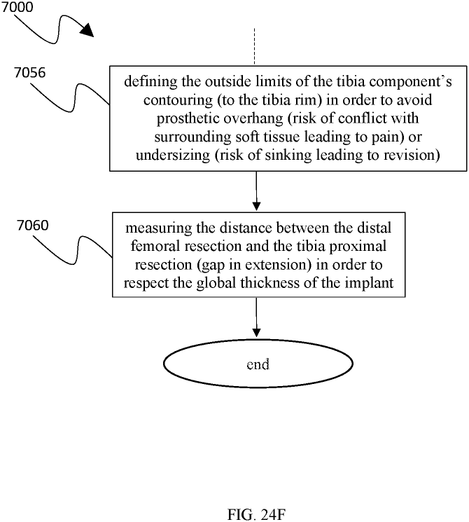

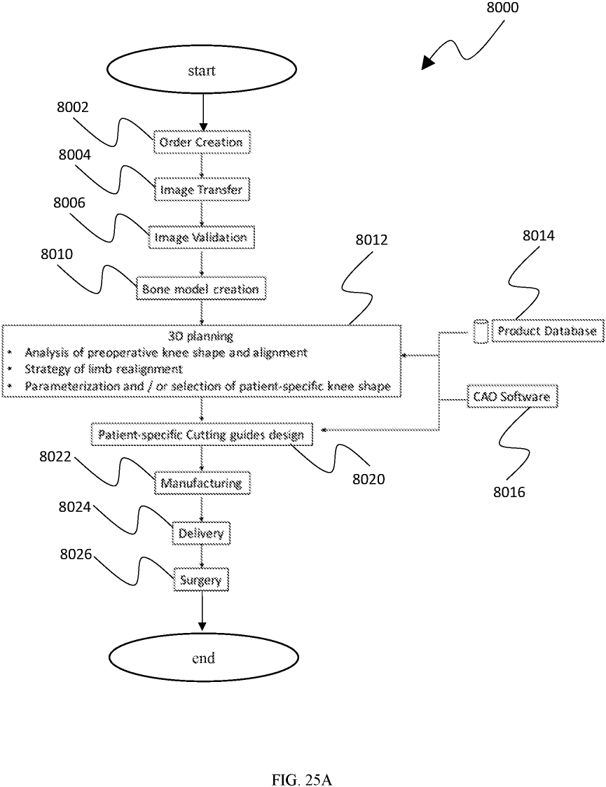

Referring now to FIGS. 24A to 24F, a method 7000 according to the invention, not necessarily in the following order. The method 7000 of making a natural, personalized prosthesis includes at least one or all of the following steps:
Step 7002: (a) measuring the preoperative, 3D HKA Alignment of the knee;
Step 7004: (b) reproducing postoperative, 3D HKA realignment to pre-arthritic HKA (if not outlier);
Step 7006: (c) defining postoperative 3D HKA realignment to corrected pre-arthritic HKA (if outlier);
Step 7010: (d) measuring the preoperative Antero-posterior dimension of the distal femur;
Step 7012: (e) reproducing the correct AP prosthetic femoral size, respecting that the implant cannot be rotated (or inclined or flexed) more than 10° in the sagittal plane ;
Step 7014: (f) measuring the preoperative FMA distal, posterior and TMA (joint lines);
Step 7016: (g) reproducing postoperative FMA, TMA obliquities to pre-arthritic FMA, TMA obliquities (if not outlier) and reproduce the pre-arthritic femoral torsion;
Step 7020: (h) defining postoperative FMA, TMA obliquities to corrected pre-arthritic FMA, TMA obliquities (if outlier) and adapt the femoral torsion according to a matrix of planning, which is an algorithm which takes for each of the three planes, anatomical inputs from landmarks (this calculates preoperative dimensions and angles defining morphotypes and phenotypes of the pathologic situation) and outputs the personalized postoperative values for the parameters defining the two types of representations/sketches for limb realignment and the knee shape (dimensions, joint surfaces);
Step 7022: (i) measuring the preoperative TL obliquity as well as depth of the trochlea;
Step 7024: (j) defining which part of the final obliquity has to be done on the bone (orientation of the resection) and which one has to be integrated into the implant (condylar offset), following rules described in the matrix of planning;
Step 7026: (k) reproducing postoperative TL obliquity to pre-arthritic TL obliquity (if not outlier) and reproduce the depth of the trochlea;
Step 7030: (l) defining postoperative TL obliquity to corrected pre-arthritic TL obliquity (if outlier) and reproduce the depth of the trochlea by adding a lateral elevation on the trochlea;
Step 7032: (m) reproducing postoperative condylar and trochlear JL curves to pre-arthritic JL curves (if not outlier);
Step 7034: (n) defining postoperative condylar and trochlear Joint Line curves to corrected pre-arthritic JL curves (if outlier by correcting the JL curve of the lateral condyle in the case of hypoplasia or of both condyles in the case of sagittal deformities for instance recurvatum or large flessum);
Step 7036: (o) reproducing postoperative condylar and trochlear Medio-Lateral curves to pre-arthritic ML (if not outlier);
Step 7040: (p) defining postoperative condylar and trochlear ML curves to corrected pre-arthritic ML curves (if outlier by correcting the ML curve of the lateral condyle or of both condyles in the case of sagittal deformities for instance recurvatum or large flessum);
Step 7042: (q) measuring the distance between the axis of each condyle to the middle of the knee and reproducing this distance;
Step 7044: (r) defining the outside limits of the articular surface for both condyles and trochlea (contouring) in order to avoid prosthetic overhang or undersizing;
Step 7046: (s) measuring the preoperative posterior tibia slope;
Step 7050: (t) reproducing the postoperative posterior tibia slope (TPS) to corrected pre-arthritic TPS if outlier;
Step 7052: (u) defining the rotation of the tibia component by measuring the angle to the anterior tibia tuberosity (TTA), which is the AP axis and by the axis going through the centers of the two circles describing the medial and lateral tibia surface geometry (ML axis);0
Step 7054: (v) defining the AP & ML position of the tibia keel to obtain a well centered keel on the tibia metaphysis and / or diaphysis;
Step 7056: (w) defining the outside limits of the tibia component’s contouring (to the tibia rim) in order to avoid prosthetic overhang (risk of conflict with surrounding soft tissue leading to pain) or undersizing (risk of sinking leading to revision); and
Step 7060: (x) measuring the distance between the distal femoral resection and the tibia proximal resection (gap in extension) in order to respect the global thickness of the implant.

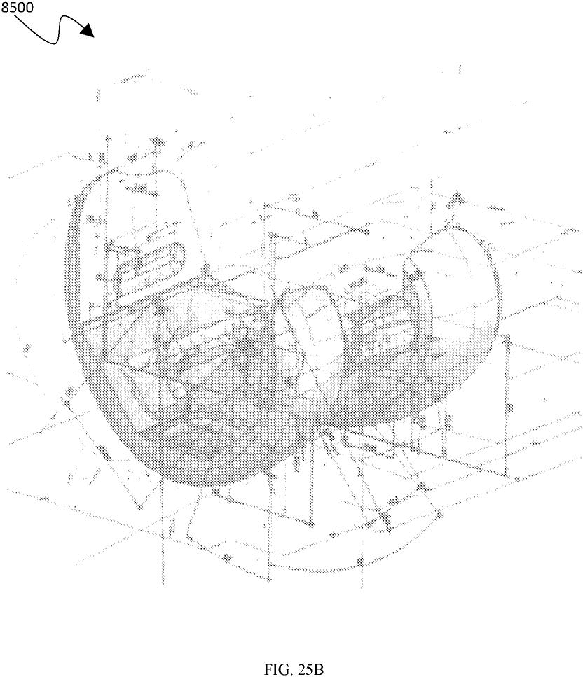

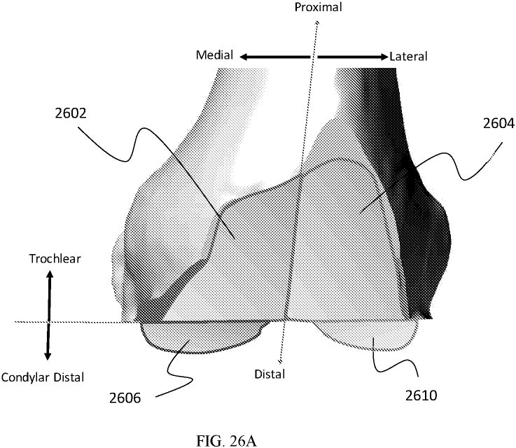

Referring now to FIG. 25A, a method 8000 according to the invention, not necessarily in the following order. The method 8000 describing different routines:
Step 8002: (a) order creation;
Step 8004: (b) image transfer;
Step 8006: (c) image validation;
Step 8010: (d) bone model creation;
Step 8012: (e) 3D planning, optionally supported by means of a product database 8014 and/or CAO Software 8016;
Step 8020: (f) patient-specific cutting guides design, optionally supported by means of a product database 8014 and/or CAO Software 8016;;
Step 8022: (g), manufacturing;
Step 8024: (h), delivery; and
Step 8026: (i), surgery.

Referring now to FIG. 25B, sketch/representation 8500 of a knee prothesis of the invention, manufactured according to the routines of FIG. 25A is shown

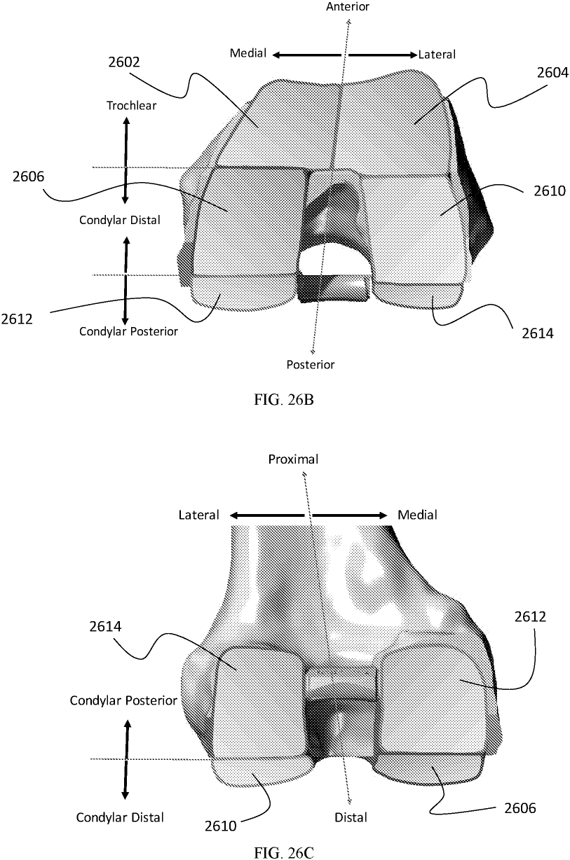

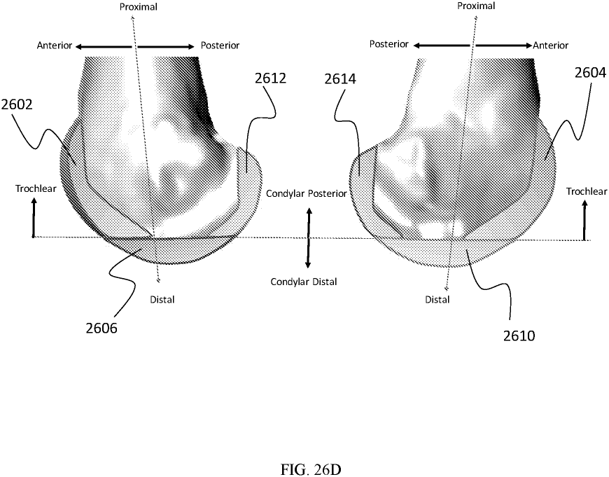

Referring now to FIGS. 26A to 26D, femoral component compartmentalization and parameterization is described in more detail. These figures show the medial anterior femoro-patellar compartment 2602, the lateral anterior femoro-patellar compartment 2604, the medial distal femoro-candylar compartment 2606, the lateral distal femoro-condylar compartment 2610, the medial posterior femoro-condylar compartment 2612 and the lateral femoro-condylar compartment 2614.

As will be appreciated by skilled artisans, the present invention may be embodied as a system, a device, or a method. In one aspect, the invention is a computer-implemented method in which a task-specific program is encoded on a medium for selecting a suitable knee prosthesis for a particular patient. The computer includes a CPU/processor, memory, an input and an output device operably interconnected thereto which implements one or more of the method steps described herein.

It should be understood that in all embodiments described herein, in which a selection is made among a number of 3D knee prostheses, a parameterized model specific to each component of the knee prosthesis (femoral component, tibial component, tibial insert, patellar component) is constructed from sketches/representations in order to generate essentially all the shape configurations that represent the worldwide anatomical variability of the normal knee joint. The parameterized models are divided into compartments (for the femoral component for instances : medial distal condylar compartment, medial posterior condylar compartment, lateral distal condylar compartment, lateral posterior condylar compartment, medial trochlear compartment, lateral trochlear compartment). In each of those independent compartments, sketches/representations are defined to reproduce the shape and dimension of a corresponding part of the knee joint surfaces and prosthetic contours. These sketches/representations are defined in each plane (coronal, sagittal and transverse) and allow for the replication in the knee prosthesis, the shape and dimension of the knee joint, in each of these planes. Fixed values as well as parameterized values (mathematical functions) define each of those sketches/representations. The parameterized values are dynamically linked to calculation tables, which define the relationships between each independent compartment and describe also the 3D variability of each of those compartments. Each prosthetic component configuration (shape and dimension) generated by the parameterized model and the calculation tables replicate the anatomical variability of the normal knee joints.

It should also be understood that in each of the substeps of the methods described herein for selecting a 3D knee prosthesis model, a planning algorithm is used which allows for the selection for each patient, among this huge family of prosthetic configurations stored in the database, the unique patient-specific component configuration, where the prosthetic shape and size best match the pre-arthritic patient knee joint motion. In a first step, anatomical landmarks are identified in the hip, knee, ankle joint (but also on upper body), in order to define the patient pathologic leg and knee morphotype and phenotype. In a second step, a 3D planning algorithm, consisting in a matrix specific to each plane, defines the specific correction to bring to the leg alignment as well as to the pathologic knee shape, in order to replicate the pre-arthritic knee alignment and knee joint shape. Note that normality (average +/- 2 standard deviations for each parameter) in a knee prothesis selection is essentially always attainable using these inputs, but outliers are not allowed and abnormal (average with more than 2 standard deviations for at least one parameter) prosthetic knee shapes will not be produced. In a third step, the algorithm will then select among all knee configurations, the unique and patient-specific prosthetic knee size, which resurface and replicate the pre-arthritic knee joint (shape and dimension), by directly positioning the component at the correct 3D orientation and alignment.

In another embodiment, a nontransient information storage medium having a knee prosthesis characterization and selection program encoded thereon is provided. When the program is executed, it implements a method which instructs a processor to aid a user in selecting a 3D knee prosthesis model for a particular patient. The method encoded thereon includes the steps of:
(a) using a parameterizing module, parameterizing a knee prosthesis according to well-defined and independent knee joint compartments,
(b) using a model generator, generating a large number knee shapes in the form of 3D knee prosthesis models which reproduce the 3D shape asymmetries of a large number of individual knee samples and storing the same on a database, thereby enabling the good replication of the knee motion of essentially any patient by generating shapes which vary the shape parameters (surface and dimension) of at least one of the compartments and storing these 3D knee prosthesis models in association with each model’s shape parameters and asymmetries in a database allowing comparing of the asymmetries of the patient’s knee with those of the 3D knee prosthesis model, and
(c) after studying the patient’s pathology and developing pre-pathological knee prosthesis criteria matching the patient’s needs, using a search module to search the database comparing the large number of knee shapes based on a best match of the asymmetries of each model to identify candidate matches:
(d) providing a display of candidate matches and their attributes on an output device;
(e) providing a means of selecting a best match among suitable candidate matches identified;
(f) optionally generating a production order if the selected knee prosthesis is not in inventory.

Where ever processing is referred to in this application, artificial intelligence such as but not limited to neuronal networks and/or machine learning algorithms may be used to facilitate the analysis of a patient’s current anatomy, to calculate most probable pre-pathological anatomies for this patient, to help select an appropriate prosthesis in the database (e.g., applying pattern recognition and classification algorithms) and/or to help design a specific prosthesis for this patient.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements such as those described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

The invention may be characterized by the following feature sets:
1. A method of selecting a 3D knee prosthesis model for a particular patient, the method consisting of:
   (a) parameterizing a knee prosthesis according to well-defined and independent knee joint compartments,
   (b) generating a large number knee shapes in the form of 3D knee prosthesis models which reproduce the 3D shape asymmetries of each individual knee, thereby enabling the replication of the knee motion of essentially any patient by generating shapes which vary the shape parameters (surface and dimension) of at least one of those compartments and storing these 3D knee prosthesis models in association with each model’s shape parameters and asymmetries in a database allowing comparing of the asymmetries of the patient’s knee with those of the 3D knee prosthesis model, and
   (c) studying the patient’s pathology and developing pre-pathological knee prosthesis criteria matching the patient’s needs,
   wherein a suitable knee prosthesis best match of the patient’s needs may be selected among the large number of knee shapes based on a best match of the asymmetries of each model, the shape selected best meeting the patient’s criteria.
2. The method of feature set 1, wherein the method includes the further step of, optionallyusing a planning algorithm, selecting a suitable knee prosthesis from among the large number of knee shapes based on a best match of the asymmetries of each model, the shape selected best meeting the patient’s criteria.
3. The method of the above feature set, wherein the selected 3D knee prosthesis model is used to produce a suitable prosthesis for the patient.
4. The method of the above feature set, wherein the produced prosthesis is made available to the surgeon for implantation.
5. The method of feature set 1, wherein invention’s design specifications for distal and posterior condyles to the trochlea part of the femoral component are used to personalize features which when put together realign a pathologic limb as it was before the pathology and reshape the joint surface as close as possible to the pre-arthritic shape and size of the knee joint.
6. A method of preparing a database of 3D knee prosthesis models from which can be selected a 3D knee prosthesis model that matches a particular patient’s needs, the method comprising the steps of:
   (a) parameterizing a selected knee prosthesis design configuration into features which correspond to well defined and independent knee joint compartments;
   (b) generating a large number of the 3D knee prosthesis models corresponding to at least one of the compartments by varying shape parameters such as surface and dimension and which reproduce the 3D shape asymmetries of a sample population of individual knees; and
   (c) populating the database with the generated models, thereby producing the database of 3D knee prosthesis models with high variability;
   (d) studying, using 3D scans, the motion of a patient’s knee;
   (e) adjusting for pathologies and optionally soft tissue influences in order to create a hypothesized pre-pathological model of the motion of the patient’s knee;
   (f) optionally using a planning algorithm, selecting from an inventory of available knee prostheses or from the database of 3D knee prosthesis models the model or models which best replicate the hypothesized pre-pathological model or models of the patient’s knee motion defined by the 3D shape asymmetries of the patient’s knee;
   (g) fabricating the selected prosthesis model if a matching knee prosthesis is not available in inventory; and
   (h) making the prosthesis available for implantation.
7. A knee prosthesis is provided which is manufactured from a 3D model selected after applying a method including the steps of:
   (a) analysis of a patient’s current and pre-pathological knee motion behavior as well as the patient’s HKA alignment,
   (b) optionally using a planning algorithm, selection of a suitable 3D model from a comprehensive database of 3D models of varying knee morphologies, each 3D model being adapted to a known morphology as well as production limitations and requirements, and
   (c) manufacturing of the selected 3D model which represents a producible and essentially custom knee prosthesis adapted to the individual patient’s 3D constitutional anatomy, thus making it possible to re-create the knee articulation as it was naturally.
8. A method according to the invention consists of the following steps:
   (a) Using CT-san, X-ray, MRI, EOS, in loaded or unloaded conditions, monopodal, bipodal, under varus / valgus stress or any other measuring equipment and/or method as well-known in the field, measuring patient’s preoperative condition including at least:
      (i) HKA alignment,
      (ii) relative movement of the femur vs tibia (sliding and rolling motion combination),
      (iii)femur and tibia contact surfaces and bone shape, and
      (iv)patella shape and position relative to the femur and tibia, and contact surfaces of the patella with the femur;
   (b) defining target postoperative HKA alignment according to (ai) and to patient’s anatomic history (if known);
   (c) defining target postoperative relative movement of the femur vs tibia (sliding and rolling motion combination) according to (b) and (aii);
   (d) defining the shape of femur and tibia prostheses contact surfaces according to (b), (c) and (aiii);
   (e) defining the shape of femur and tibia prostheses attachment according to (d) and (aiii);
   (f) defining target postoperative patella position relative to the femur prosthesis and relative to the tibia prosthesis according to (b), (c), (d), (aiv) and to patient’s anatomic history (if known);
   (g) defining the shape of the contact surfaces between femur prosthesis and patella according to (b), (c), (d), (f) and (aiv) (Contact surface only between the femoral component and the patella component, not between the patella component and the tibia component); and
   (h) merging all the above definitions into an individually adapted knee prosthesis.
9. The method of one of feature sets 1 to 4 wherein the 3D scans are video scans of the patient’s knee in motion.
10. The method of one of feature sets 1 to 4, wherein the known joint compartments include at least one of the following compartments: extension compartment, flexion compartment; medial compartment; lateral compartment; femoro-tibia compartment; and femoro-patellar compartment.
11. The of one of feature sets 1 to 4, wherein the method includes consideration of the articulation of the patella in selecting a suitable 3D model.
12. A prosthesis made according of one of feature sets 1 to 4.
13. The method of one of feature sets 1 to 4, wherein the knee prosthesis of the invention is adapted in the case of constitutional femur varus such that the femoral prosthesis (1110) has the distal lateral condyle shorter than the distal medial condyle, generating an offset (1172) between the condyles, the same offset (1172) is reproduced in the tibial insert component (1160).
14. The method of one of feature sets 1 to 4, wherein the knee prosthesis of the invention is adapted for a case of constitutional femur valgus where the femoral prosthesis (1110) has the distal lateral condyle longer than the distal medial condyle generating an offset (1174) between the condyles, the same offset (1174) is reproduced in the tibial insert component (1160).
15. The method of one of feature sets 1 to 4, wherein the knee prosthesis of the invention is adapted for a case of constitutional femur varus where the femoral prosthesis (1110) has an angle (1182) opening from the distal lateral condyle to the distal medial condyle, the same angle (1182) is reproduced in the tibial insert component (1160).
16. The method of one of feature sets 1 to 4, wherein the knee prosthesis of the invention is adapted for a case of constitutional femur valgus where the femoral prosthesis (1110) has an angle (1184) opening from the distal medial condyle to the distal lateral condyle, the same angle (1184) is reproduced in the tibial insert component (1160).
17. The method of one of feature sets 1 to 4, wherein the knee prosthesis of the invention, the offsets (1172, 1174) vary from 0 to 10 mm, and the angles (1182, 1184) vary from 0° to 15°.
18. The method of one of feature sets 1 to 4, wherein, when sizing the prosthesis, the shape radius of the medial condyle surface in the coronal and transverse plane (962), the shape radius of the trochlea surface in the coronal plane (963) and the shape radius of the lateral condyle surface in the coronal and transverse plane (964) are adjusted independently from each other and independently from the prosthesis size and from the patient’s hip-knee-ankle (HKA) alignment, be it normal, varus or valgus.
19. The method of one of feature sets 1 to 4, wherein contouring (outside dimension of the prosthesis) is adjusted so as not to overhang or uncover the resected area to resurface.
20. The method of one of feature sets 1 to 4, wherein contouring angles (982, 984, 992, 994) for the anterior and posterior faces of the femoral prosthesis are adjusted independently from each other and independently from the prosthesis size and from the patient’s hip-knee-ankle (HKA) alignment, be it normal, varus or valgus.
21. The method of one of feature sets 1 to 4, wherein contouring angles (982, 984, 992, 994) for the anterior and posterior faces of the femoral prosthesis are varied from 0° to 50° in order to obtain the desired knee kinematics and to adapt to the patient’s size.
22. The method of one of feature sets 1 to 4, wherein the shape of the medial condyle surface in the sagittal plane (medial condyle J-curve) (972), the shape of the trochlea surface in the sagittal plane (trochlea J-curve) (973) and the shape of the lateral condyle surface in the sagittal plane (lateral condyle J-curve) (974) are adjusted independently from each other and independently from the prosthesis size and from the patient’s hip-knee-ankle (HKA) alignment, be it normal, varus or valgus.
23. The method of one of feature sets 1 to 4, wherein the shape of the medial condyle surface in the sagittal plane (medial condyle J-curve) (972), the shape of the trochlea surface in the sagittal plane (trochlea J-curve) (973) and the shape of the lateral condyle surface in the sagittal plane (lateral condyle J-curve) (974) can be made of one radius but can also be made of a combination of two or more radii, which dimension can vary from 15 mm to 65 mm.
24. The method of any of the above feature sets, wherein via use of a combination of radii, the transition from one radius to the next radius is smoothened by the use of a spline or any other appropriate curve.
25. The method of any of the above feature sets, wherein the femoral part of the knee prosthesis of the invention is adapted to any bony resections selected from one of the group of resections consisting of planar, inclined, curved, offset, and double-inclined adapted to the individual need of the patient.
26. The method of the above feature set, wherein the bone resection is sawed or milled so as to be adapted to the individual needs of the patient.
27. The method of making a natural, personalized implant including at least one or all of the following steps:
   (a) measuring the preoperative, 3D HKA Alignment of the knee;
   (b) reproducing postoperative, 3D HKA realignment to pre-arthritic HKA (if not outlier);
   (c) defining postoperative 3D HKA realignment to corrected pre-arthritic HKA (if outlier);
   (d) measuring the preoperative Antero-posterior dimension of the distal femur;
   (e) reproducing the correct AP prosthetic femoral size, respecting that the implant cannot be rotated (or inclined or flexed) more than 10° in the sagittal plane ;
   (f) measuring the preoperative FMA distal, posterior and TMA (joint lines);
   (g) reproducing postoperative FMA, TMA obliquities to pre-arthritic FMA, TMA obliquities (if not outlier) and reproduce the pre-arthritic femoral torsion;
   (h) defining postoperative FMA, TMA obliquities to corrected pre-arthritic FMA, TMA obliquities (if outlier) and adapt the femoral torsion according to the matrix of planning;
   (i) measuring the preoperative TL obliquity as well as depth of the trochlea;
   (j) defining which part of the final obliquity has to be done on the bone (orientation of the resection) and which one has to be integrated into the implant (condylar offset), following rules described in the matrix of planning;
   (k) reproducing postoperative TL obliquity to pre-arthritic TL obliquity (if not outlier) and reproduce the depth of the trochlea;
   (l) defining postoperative TL obliquity to corrected pre-arthritic TL obliquity (if outlier) and reproduce the depth of the trochlea by adding a lateral elevation on the trochlea;
   (m) reproducing postoperative condylar and trochlear JL curves to pre-arthritic JL curves (if not outlier);
   (n) defining postoperative condylar and trochlear Joint Line curves to corrected pre-arthritic JL curves (if outlier by correcting the JL curve of the lateral condyle in the case of hypoplasia or of both condyles in the case of sagittal deformities for instance recurvatum or large flessum);
   (o) reproducing postoperative condylar and trochlear Medio-Lateral curves to pre-arthritic ML (if not outlier);
   (p) defining postoperative condylar and trochlear ML curves to corrected pre-arthritic ML curves (if outlier by correcting the ML curve of the lateral condyle or of both condyles in the case of sagittal deformities for instance recurvatum or large flessum);
   (q) measuring the distance between the axis of each condyle to the middle of the knee and reproducing the distance;
   (r) defining the outside limits of the articular surface for both condyles and trochlea (contouring) in order to avoid prosthetic overhang or undersizing;
   (s) measuring the preoperative posterior tibia slope;
   (t) reproducing the postoperative posterior tibia slope (TPS) to corrected pre-arthritic TPS if outlier;
   (u) defining the rotation of the tibia component by measuring the angle to the anterior tibia tuberosity (TTA), which is the AP axis and by the axis going through the centers of the two circles describing the medial and lateral tibia surface geometry (ML axis);0
   (v) defining the AP & ML position of the tibia keel to obtain a well centered keel on the tibia metaphysis and / or diaphysis;
   (w) defining the outside limits of the tibia component’s contouring (to the tibia rim) in order to avoid prosthetic overhang (risk of conflict with surrounding soft tissue leading to pain) or undersizing (risk of sinking leading to revision); and
   (x) measuring the distance between the distal femoral resection and the tibia proximal resection (gap in extension) in order to respect the global thickness of the implant.
28. A prosthesis made according of one of feature sets 1 to 4, wherein the tibial insert, the tibial tray and the keel, components of the prosthesis of the invention, are each made up of one or more parts and assembled before or during the surgery.
29. The prosthesis of the above feature set, wherein at least one of these components has
   (a) one element selected from one of the group of elements consisting of
      (i) the articulation surface of the tibial insert comprising the medial condyle corresponding surface (1262),
      (ii) the trochlea corresponding surface (1263), and
      (iii) the lateral condyle corresponding surface (1263),
   (b) a bone facing surface of the tibial tray (1230), and
   (c) the keel portion (1240).
30. The prosthesis of the above feature set, wherein any desired orientation angle, offset or any combination thereof may be applied to the tibial insert, the tibial tray and the keel so as to best adapt to the need of the individual patient.
31. The prosthesis of either of the above two feature sets, wherein the tibial insert, the tibial tray and the keel can each be formed of one or more elements.
32. The prosthesis of any of the above feature sets, wherein the keel (1240) is formed so as not to be orthogonal to the bone facing surface (1230), but is oriented at a selected angle (1242) so as to adapt to the need of the individual patient.
33. The prosthesis of any of the above feature sets, wherein the keel (1240) is not orthogonal to the bone facing surface (1230), but is oriented at an angle (1242), and the bone facing surface (1230) is oriented at an angle (1232) so as to adapt to the need of the individual patient.
34. The prosthesis of the above feature set, wherein the medial insert thickness is made thin so as to favor the knee to be orientated in varus.
35. The prosthesis of any of the above feature sets, wherein the keel (1240) is not formed orthogonal to the bone facing surface (1230), but is oriented at a selected angle (1242), the bone facing surface (1230) is oriented at another selected angle (1232), and the lateral condyle corresponding surface (1264) presents an offset (1265), so as to adapt to the need of the individual patient.
36. The prosthesis of any of the above feature sets, wherein the keel (1240) is not orthogonal to the bone facing surface (1230), but is oriented at a selected angle (1242), the bone facing surface (1230) is oriented at a second selected angle (1232), and the bicondyle distal tangent (1266) is oriented at a third selected angle (1267), so as to adapt to the need of the individual patient.
37. The prosthesis of any of the above feature sets, wherein the offset (1265) can vary from 0 to 10 mm, and the orientation angles (1232, 1242, 1267) can vary up to 12°.
38. The prosthesis of the above feature set wherein the offset can vary from -10° to +10° in mediolateral or in anteroposterior dimensions, and can be oriented up to 12° around the longitudinal axis of the keel.
39. The prosthesis of any of the above feature sets, wherein the sagittal J-curve (1310) is adapted to fit with the corresponding surface of the femoral prosthesis of the invention, so that the functioning of the knee prosthesis of the invention fits the need of the individual patient.
40. The prosthesis of the above feature set wherein the sagittal J-curve (1310) is essentially a single radius.
41. The prosthesis of the penultimate feature set, wherein the sagittal J-curve (1310) is essentially a combination of two or more radii (1312, 1313, 1314, 1315) , which dimension falls in the range from 15 mm to 80 mm.
42. The prosthesis of any of the above feature sets, wherein, to fit with the tibia of the individual patient, in the sagittal plane, the keel (1340) of the tibial component (1300) of a knee prosthesis of the invention is placed at the center of the tibial component (1300) and optionally presents an offset (1342 towards the anterior part of the tibia or towards the posterior part of the tibia.
43. The prosthesis of the above feature set, wherein the offset (1342) can vary from 0 to 10 mm.
44. The prosthesis of the penultimate feature set, wherein the offset varies from -10° to +10° in mediolateral or in anteroposterior dimensions, and is oriented up to 12° around the longitudinal axis of the keel.
45. The prosthesis of the above feature set, wherein, for the same purpose of fitting with the tibia of the individual patient, in the sagittal plane, the bone facing surface (1332) of the tibial component (1300) of a knee prosthesis of the invention can be oriented at a selected angle (1344) which may vary up to 12°.
46. The prosthesis of any of the above feature sets, wherein the articulating surfaces of the patella component (1862, 1863, 1864) are made to fit to their respective corresponding surfaces of the femoral component (1852, 1853, 1854), also taking into account the patient’s hip-knee-ankle (HKA) alignment, be it normal, varus or valgus.
47. The prosthesis of any of the above feature sets, wherein, in the case the femoral component has an offset (1872, 1874) between the distal lateral condyle and the distal medial condyle, the offset (1872, 1874) is reproduced in the patella component.
48. The prosthesis of the above feature set, wherein the patella component of a knee prosthesis of the invention, the mediolateral surfaces can be symmetrical or asymmetrical between medial and lateral compartment, and the anteroposterior surfaces can be symmetrical or asymmetrical between the anterior and posterior compartment and selected, optionally using a planning algorithm, so as to match the needs of the individual patient.
49. The prosthesis of any of the above feature sets, wherein each of the medial, lateral, anterior, posterior compartments are specified to fall in the range from 8 mm to 30 mm in width and height, which values are independent of the patella thickness, which should be at least 6 mm or more.
50. The method of any of the above feature sets wherein curve/surface fitting and smoothing techniques are applied between shapes that interact across adjacent bone compartments in order to meld elements of the prosthesis that correspond to a bone compartment in order to create a composite knee prosthesis adapted to the needs of the patient.
51. A partial or total knee prosthesis designed according to an individual patient’s constitutional anatomy, re-creating the knee articulation as it was naturally wherein its geometry is determined by varying independently to each other, the medial femoro-tibial joint to the lateral femoro-tibial joint to the femoro-patellar joint.
52. A method for production of a partial or total knee prosthesis adapted to an individual patient’s constitutional anatomy, the method comprises a design step considering the current and the pre-pathology knee motion behavior of the patient, and further considering his individual Hip-Knee-Ankle (HKA) alignment, and using these inputs, re-creating a knee articulation model as it was naturally, wherein further, this re-created natural knee articulation model and not the pathological knee articulation model is used to create a prostheses that re-creates this natural knee articulation.
53. A femoral prosthesis for implantation on a femur of a patient’s knee, comprising:
   two condylar portions comprising the medial and lateral condyles, having a bone-facing surface for abutting at least a portion of each condyle of the patient’s knee and an articular surface generally opposite each bone-facing surface; each articular surface having a curvature (J-curve) generally disposed in a first plane (sagittal plane) and ML curve generally disposed in a second and third plane (frontal plane for the distal condyles and transverse planes for the posterior condyles); each articular surfaces of the medial and lateral condyles may have a condylar offset in the second and third plane, which can be equivalent or not;
   a trochlear portion, comprising the trochlear depth as well as the lateral and medial trochlear elevations, having a bone-facing surface for abutting at least a portion of the trochlea of the patient’s knee and an articular surface generally opposite the bone-facing surface; each articular surface having a curvature (J-curve) generally disposed in a first plane (sagittal plane) and ML curve generally disposed in a second and third plane (frontal plane and transverse planes); each articular surfaces of the medial and lateral elevations may have an offset and a depth to the trochlea in the second and third plane, which can be equivalent or not;
   the articular surface orientation of the trochlea portion to the distal and posterior condylar portions of the distal and posterior condyles are not dependent and can be parallel or obliquely oriented (convergent or divergent) in at least one of the planes;
   an ML condylar offset can be integrated between the medial and lateral articular surfaces of the distal (= distal condylar offset) and posterior (= posterior condylar offset) condylar portions of the distal and posterior condyles, this condylar offset being equivalent or different between distal and posterior condylar portions; and
   an ML trochlear offset can be integrated between the medial and lateral articular surfaces of the medial and lateral trochlear elevation, this trochlear offset being equivalent or different than the condylar offset of the distal and posterior condyles.
54. The prosthesis of the above feature set, wherein the sagittal J-curve of at least one of the joint-surface from the distal and posterior condyles (medial, lateral) or the trochlea (elevation, trochlea depth) is defined by a single, double or multi-radius or is fitting with a patient-specific J-curve.
55. The prosthesis of one of the above two feature sets, wherein the sagittal J-curve of at least one of the medial and lateral joint-surfaces from the distal and posterior condyles is positioned at a fixed or variable distance to the trochlea J-curves (medial and / or lateral elevation, trochlea depth), symmetrically or asymmetrically.
56. The prosthesis of one of feature sets 51 or 52, wherein the sagittal J-curve of at least one of the joint-surface from the distal and posterior condyles (medial, lateral = narrowing angle) or the trochlea (medial and / or lateral elevation, trochlea depth = sulcus axis in frontal plane, Whiteside line in the axial plane) is parallel or obliquely oriented in at least one of the planes, mainly the frontal and the axial planes.
57. The prosthesis of feature sets 51 to 54, wherein at least one of the joint facing-surface of the condylar portion and / or of the trochlea portion has an articular geometry and dimensions corresponding (or close matching, or close fitting) to the patient’s knee articular surface, in terms of sizing (comprising at least AP sizing), shape (comprising at least condylar and trochlear offset, J-curves and ML curves) and contour (comprising at least AP/ML, sizing, narrowing angle, trochlear height, posterior condyles height).
58. The prosthesis of feature sets 51 to 55, wherein the tangent linking the medial and lateral most distal points of the bone-facing surfaces of the distal or the tangent linking the medial and lateral most posterior points of the bone-facing surfaces of the posterior condyles or the tangent linking the medial and lateral most anterior points of the bone-facing surfaces of trochlea portion are parallel or oblique between themselves.
59. The prosthesis of any of the above prosthesis feature sets, wherein the bone-facing surfaces are defined with a single straight flat or oblique surface or with two staggered (offset) flat or oblique surfaces or with a staggered (offset) curved surface.
60. The prosthesis of any of the above prosthesis feature sets, wherein the bone-facing surfaces can be fixed to the bone with a cemented or cementless fixation.
61. The prosthesis of any of the above prosthesis feature sets, wherein the prosthesis corresponds to different systems (PS: Postero-Stabilized, UC: Ultra-Congruent, PCR: Posterior Cruciate Retaining, BCR: Bi-Cruciate Retaining), for mobile insert or fixed insert, for primary or revision knee (semi-constrained or constrained, hinged), for cemented or cementless or any other type of fixation, for monobloc or modular components, for every material (Ti, CrCo, Ceramic, ...)
62. A method of manufacturing a knee prosthesis from a 3D model selected after applying a method, the method including:
   (a) analysis of a patient’s current and pre-pathological knee motion behavior as well as the patient’s 3D HKA alignment,
   (b) optionally using a planning algorithm, selection of a suitable 3D model from a comprehensive database of 3D models of varying knee morphologies, each 3D model being adapted to a known morphology as well as production limitations and requirements,
   (c) manufacturing of the selected 3D model which represents a producible and essentially custom knee prosthesis adapted to the individual patient’s 3D constitutional anatomy, thus making it possible to re-create the knee articulation as it was naturally.
63. A nontransient information storage medium having a knee prosthesis characterization and selection program that instructs a processor to implement one of any of the above methods so as to accept inputs and produce outputs.
64. A nontransient information storage medium having a knee prosthesis characterization and selection program encoded thereon that, when executed, implements a method which instructs a processor execute steps which aid a user in selecting a 3D knee prosthesis model for a particular patient, the method consisting of the steps of:
   (a) parameterizing a knee prosthesis according to well-defined and independent knee joint compartments,
   (b) generating a large number knee shapes in the form of 3D knee prosthesis models which reproduce the 3D shape asymmetries of a large number of individual knee samples including models that replicate the knee motion of essentially any patient by generating shapes which vary the shape parameters (surface and dimension) of at least one of the compartments and storing these 3D knee prosthesis models in association with each model’s shape parameters and asymmetries in a database allowing comparing of the asymmetries of the patient’s knee with those of the 3D knee prosthesis model, and
   (c) after studying the patient’s pathology and developing pre-pathological knee prosthesis criteria matching the patient’s needs, optionally using a planning algorithm, searching the database comparing the large number of knee shapes based on a best match of the asymmetries of each model to identify candidate matches:
   (d) providing a display of candidate matches and their attributes on an output device;
   (e) providing a means of selecting a best match among suitable candidate matches identified;
   (f) optionally generating a production order if the selected knee prosthesis is not in inventory.
65. The medium of the above feature set, wherein the processor is a computer processor connected to memory, wherein the processor responds to the program to access a database adapted to store 3D knee prosthesis models or inventoried knee prostheses.
66. The medium of the above feature set, wherein the processor responds to inputs and outputs communicated by the program to and from a user.

Additional features and functionality of the invention are described in the claims appended hereto and/or in the abstract. Such claims and/or abstract are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

### Appendix

### Definitions

Definitions of Knee anatomical points
Definitions of Knee center
Definitions of the Average Knee Flexion Axis
Definitions of Knee Alignment
Coronal or frontal Plane
Axial or Transverse Plane

### Observations

Comparison with Standard (Off-the-shelf) knee prostheses and limits of this system Description of Knee prosthesis

### Description of the Invention

Difference between STD (Off-The Shelf) prosthesis and our Personalized knee prosthesis:

Definitions of Knee anatomical points:
FHC (332): Femoral Head Center
KC (330): Knee Center
TC (334): Talus Center (not represented)
ME (342): Medial Epicondyle
LE (344): Lateral Epicondyle
MDC (352): Medial Distal Condyle
LDC (354): Lateral Distal Condyle
MPC (362): Medial Posterior Condyle
LPC (364): Lateral Posterior Condyle
TGH (372): Trochlea Groove High
TGL (374): Trochlea Groove Low
LT (384): Lateral Elevation of the Trochlea
MT (382): Medial Elevation of the Trochlea

Definitions of Knee center:
KC (330): Top of the intercondylar notch
TSE (3_): Top of the tibia eminencia (not represented)
MTEA (3_): Middle of the TEA (346)

Definitions of the Average Knee Flexion Axis:
TEAs (3464): Surgical Transepicondylar Axis (top of LE (344) to sulcus of ME (342))
TEAc (3462): Clinical Transepicondylar Axis (top of LE (344) to top of ME (342))
CA: Cylinder axis (Links the center of the two spheres of the posterior condyles)
EFA: Extension Facet Axis (links the center of distal radius of the two condyles)
FFA: Flexion Facet Axis (links the center of posterior radius of the two condyles)
FHA: Femoral Helical Axis (combines flexion axis with axial rotation)
   With the mechanism of 4-bar linkages, the average knee flexion axis could also be at the intersection of the two cruciate ligaments in the sagittal plane, or at the intersection of both the cruciate and collateral ligaments.
   With Kinematics alignment, BCD and BCP are the instantaneous knee flexion axes.

Definitions of Knee Alignment:
HKA: Mechanical Axis of the Limb
(=load bearing axis)
Links two lines, the first between FHC to KC with the line between KC and TC
If the angle between the two line is 0 (HKA 180°), the alignment is neutral
If the angle between the two line is > 0 (HKA > 180°), the alignment is valgus
If the angle between the two line is < 0 (HKA < 180°), the alignment is varus

Coronal or frontal Plane:
FAA (326): Femoral Anatomical Axis (links KC (330) to middle of diaphysis)
FMA (336): Femoral Mechanical Axis (links FHC (332) to KC (330))
HKS (338): Angle between FAA (326) & FMA (336)
   Range is between 1° to 10°
BCD (356): Bicondyle Distal Tangent (links LDC (354) to MDC (352))
Alpha (α, 358): Angle between FMA (336) and BCD (356)
   Also called FMA: femoral Mechanical Angle (although to an unskilled reader, there may be a risk of confusion between FMA= Femoral Mechanical Axis (336) and FMA=Femoral Mechanical Angle, a person of ordinary skill will know the difference from its context).
   Also called midface: mechanical Medial Distal Femoral Angle
   Can be orthogonal or not to FMA (336)
   Range is between 82° to 105°
TEA (346): Transepicondylar Axis or Biepicondylar Axis
   Links ME (342) to LE (344)
   TEAs (3464): Surgical Transepicondylar Axis
   TEAc (3462): Clinical Transepicondylar Axis
   = Insertions of the two collateral ligaments
   Can be parallel or not to BCD (356)
   Can be orthogonal or not to FMA (336)
DCA (358): Distal Condylar Angle
   Angle between TEA (346) and BCD (356)
   Range is between -5° to 10°
SA (376): Sulcus Axis (links KC (330) to TGH (372))
   SA can be between FMA (336) and FAA (326) or outside the range
   Can be orthogonal or not to TEA (346) and / or to BCD (356)

Axial or Transverse Plane:
TL (386): Trochlear Line, links LT (382) to MT (384)
TEA (346): Transepicondylar Axis or Biepicondylar Axis, links ME (342) to LE (344)
BCP (366): Bicondyle Posterior Tangent (links LPC (364) to MPC (362))
PCA (368): Posterior Condylar Angle
   Angle between TEA (346) and BCP (366)
   Range is between -5° to 10°
WL (377): Whiteside Line, links KC (330) to TGL (374)
   WL can be orthogonal or not to TEA and / or to BCP
ATA (388): Anterior Trochlear Axis
   Angle between TEA (346) and TL (386)
   Range is between -5° to 10°

1-69. (canceled)

70. A computer-assisted method of making a natural, personalized implant for a patient including the following steps:
(a) measuring the patient’s preoperative condition including at least;
(i) HKA alignment,
(ii) relative movement of the femur vs tibia, namely sliding and rolling motion combination,
(iii) femur and tibia contact surfaces and bone shape, and
(iv) patella shape and position relative to the femur and tibia, and contact surfaces of the patella with the femur;
(b) reproducing postoperative, 3D HKA realignment to pre-arthritic HKA if not outlier;
(_{C}) defining postoperative 3D HKA realignment to corrected pre-arthritic HKA HKA if outlier;
(d) defining, target postoperative relative movement of the femur vs tibia (sliding and rolling motion combination) according to (b), (c) and (a)(ii);
(e) defining the shape of femur and tibia prosthesis contact surfaces according to (b), (c) and (a)(iii);
(f) defining the shape of femur and tibia prostheses attachment according to (e) and (a)(iii);
(g) defining target postoperative patella position relative to the femur prosthesis and relative to the tibia prosthesis according to (b), (c), (d), (e), (a)(iv) and to patient’s anatomic history (if known);
(h) defining the shape of the contact surfaces between femur prosthesis and patella according to (b), (c), (d), (e), (g), and (a)(iv) (Contact surface only between the femoral component and the patella component, not between the patella component and the tibia component);
(i) making other measurements depending on the needs of the patient; and
(J) fabricating the prosthesis according to the measurements and definitions.

71. The method of claim 70, further including the step of measuring the preoperative Antero-posterior dimension of the distal femur and reproducing the correct AP prosthetic femoral size, respecting that the implant cannot be rotated (or inclined or flexed) more than 10° in the sagittal plane.

72. The method of claim 70, further including the step of measuring the preoperative FMA distal, posterior and TMA (joint lines); reproducing postoperative FMA, IMA obliquities to pre-arthritic FMA, TMA obliquities, if not outlier, and reproducing the pre-arthritic femoral torsion, and defining postoperative FMA, TMA obliquities to corrected pre-arthritic FMA, TMA obliquities, if outlier, and adapting the femoral torsion according to the matrix of planning.

73. The method of claim 70, further including the step of measuring the preoperative TL obliquity as well as depth of the trochlea; and defining which part of the final obliquity has to be done on the bone, namely orientation of the resection, and which one has to be integrated into the implant, (condylar offset), following rules described in the matrix of planning.

74. The method of claim 70, further including the step of either reproducing postoperative TL obliquity to pre-arthritic TL obliquity (if not outlier) and reproducing the depth of the trochlea; or defining postoperative TL obliquity to corrected pre-arthritic TL obliquity, if outlier, and reproducing the depth of the trochlea by adding a lateral elevation on the trochlea.

75. The method of claim 70, further including the step of either reproducing postoperative condylar and trochlear JL curves to pre-arthritic JL curves, if not outlier or defining postoperative condylar and trochlear Joint Line curves to corrected pre-artritic JL curves, if outlier by correcting the JL curve of the lateral condyle in the case of hypoplasia or of both condyles in the case of sagittal deformities for instance recurvatum or large flessum.

76. The method of claim 70, further including the step of either reproducing postoperative condylar and trochlear Medio-Lateral curves to pre-arthritic ML (if not outlier) or defining postoperative condylar and trochlear ML curves to corrected pre-arthritic ML curves, if outlier by correcting the ML curve of the lateral condyle or of both condyles in the case of sagittal deformities for instance recurvatum or large flessum.

77. The method of claim 70, further including the step of measuring the distance between the axis of each condyle to the middle of the knee and reproducing the distance.

78. The method of claim 70, further including the step of defining the outside limits of the articular surface for both condyles and trochlea (contouring) in order to avoid prosthetic overhang or undersizing.

79. The method of claim 70, further including the step of measuring the preoperative posterior tibia slope; and either reproducing the postoperative posterior tibia slope (TPS) to corrected pre-arthritic TPS if not outlier or defining postoperative posterior tibia slope to be corrected to pre-arthritic posterior tibia slope if outlier.

80. The method of claim 70, further including the step of defining the rotation of the tibia component by measuring the angle to the anterior tibia tuberosity (TTA), which is the AP axis and by the axis going through the centers of the two circles describing the medial and lateral tibia surface geometry (ML axis).

81. The method of claim 70, further including the step of defining the AP & ML position of the tibia keel to obtain a well centered keel on the tibia metaphysis and / or diaphysis.

82. The method of claim 70, further including the step of defining the outside limits of the tibia components contouring (to the tibia rim) in order to avoid prosthetic overhang (risk of conflict with surrounding soft tissue leading to pain) or undersizing (risk of sinking leading to revision).

83. The method of claim 70, further including the step of measuring the distance between the distal femoral resection and the tibia proximal resection (gap in extension) in order to respect the global thickness of the implant.

84. A prosthesis made according to claim 70, wherein the tibial insert, the tibial tray and the keel, components of the prosthesis of the invention, are each made up of one or more parts which are adapted to be assembled before or during the surgery.

85. The prosthesis of the claim immediately above, wherein at least one of these components has
(a) one element selected from one of the group of elements consisting of
(i) the articulation surface of the tibial insert comprising the medial condyle corresponding surface.
(ii) the trochlea corresponding surface, and
(iii) the lateral condyle corresponding surface,
(b) a bone facing surface of the tibial tray, and
(c) the keel portion.

86. The prosthesis of the claim immediately above, wherein any desired orientation angle, offset or any combination thereof may be applied to the tibial insert, the tibial tray and the keel so as to best adapt to the need of the individual patient.

87. The prosthesis of claim 85, wherein the tibial insert, the tibial tray and the keel are each formed of one or more elements.

88. The prosthesis of claim 84, wherein the keel is formed so as not to be orthogonal to the bone facing surface, and is oriented at a selected angle so as to adapt to the need of the individual patient.

89. The prosthesis of claim 84, wherein the keel is not orthogonal to the bone facing surface, and is oriented at another angle, and the bone facing surface is oriented at an angle so as to adapt to the need of the individual patient.

90. The prosthesis of the claim immediately above, wherein the medial insert thickness is thin so as to favor the knee to be orientated in varus.

91. The prosthesis of claim 84, wherein the keel is not formed orthogonal to the bone facing surface, and is oriented at a selected angle, the bone facing surface is oriented at another selected angle, and the lateral condyle corresponding surface presents an offset, so as to adapt to the need of the individual patient.

92. The prosthesis of claim 84, wherein the keel is not orthogonal to the bone facing surface, and is oriented at a selected angle, the bone facing surface is oriented at a second selected angle, and the bicondyle distal tangent is oriented at a third selected angle, so as to adapt to the need of the individual patient.

93. The prosthesis of claim 84, wherein the offset is selected from within the range of from 0 to 10 mm, and the orientation angles of the offset angles are selected up to 12°.

94. The prosthesis of the claim immediately above wherein the offset is selected from within a range from -10° to + 10° in mediolateral or in anteroposterior dimensions, and is oriented at an angle of up to 12° around the longitudinal axis of the keel.

95. The prosthesis of claim 84, wherein the sagittal J-curve is adapted to fit with the corresponding surface of the femoral prosthesis, so that the functioning of the knee prosthesis of the invention fits the need of the individual patient.

96. The prosthesis of the claim immediately above wherein the sagittal J-curve is essentially a single radius.

97. The prosthesis of claim 95, wherein the sagittal J-curve is essentially a combination of two or more radii, which dimension falls in the range from 15 mm to 80 mm.

98. The prosthesis of claim 84, wherein, to fit with the tibia of the individual patient, in the sagittal plane, the keel of the tibial component of a knee prosthesis of the invention is placed at the center of the tibial component and optionally presents an offset towards the anterior part of the tibia or towards the posterior part of the tibia.

99. The prosthesis of the claim immediately above, wherein the offset is selected from a range of from 0 to 10 mm.

100. The prosthesis of claim 98, wherein the offset is selected from within the range of -10° to +10° in mediolateral or in anteroposterior dimensions, and is oriented at an angle selected within the range of up to 12° around the longitudinal axis of the keel.

101. The prosthesis of the claim immediately above, wherein, for the same purpose of fitting with the tibia of the individual patient, in the sagittal plane, the bone facing surface of the tibial component of a knee prosthesis of the invention is oriented at an angle selected from within a range which varies from 0° to 12°.

102. The prosthesis of claim 84, wherein the articulating surfaces of the patella component are made to fit to their respective corresponding surfaces of the femoral component, also taking into account the patient’s hip-knee-ankle (HKA) alignment, be it normal, varus or valgus.

103. The prosthesis of claim 84, wherein, in the case the femoral component has an offset between the distal lateral condyle and the distal medial condyle, the offset is reproduced in the patella component.

104. The prosthesis of the claim immediately above, wherein the patella component and the mediolateral surfaces can be symmetrical or asymmetrical between medial and lateral compartments, and the anteroposterior surfaces can be symmetrical or asymmetrical between the anterior and posterior compartment and selected, optionally using a planning algorithm, so as to match the needs of the individual patient.

105. The prosthesis of claim 84, wherein each of the medial, lateral, anterior, posterior compartments are formed so as to fall in the range from 8 mm to 30 mm in width and height, which values are independent of the patella thickness, which thickness is selected to be at least 6 mm or more.

106. The method of any of claim 70, the method including the step of applying curve/surface fitting and smoothing techniques between shapes that interact across adjacent bone compartments in order to meld elements of the prosthesis that correspond to a bone compartment thereby creating a composite knee prosthesis adapted to the needs of the patient.

107. A method for production of a partial or total knee prosthesis adapted to an individual patient’s constitutional anatomy, the method comprises a design step considering the current and the pre-pathology knee motion behavior of the patient, and further considering the patient’s individual Hip-Knee-Ankle (HKA) alignment, and using these inputs, re-creating a knee articulation model as it was naturally, wherein further, this re-created natural knee articulation model and not the pathological knee articulation model is used to create a prostheses that re-creates this natural knee articulation.

108. A femoral prosthesis for implantation on a femur of a patient’s knee, comprising:
(a) two condylar portions comprising the medial and lateral condyles, having a bone-facing surface for abutting at least a portion of each condyle of the patient’s knee and an articular surface generally opposite each bone-facing surface, each articular surface having a curvature (J-curve) generally disposed in a first plane (sagittal plane) and ML curve generally disposed in a second and third plane (frontal plane for the distal condyles and transverse planes for the posterior condyles); each articular surfaces of the medial and lateral condyles may have a condylar offset in the second and third plane, which is optionally equivalent;
(b) a trochlear portion, comprising the trochlear depth as well as the lateral and medial trochlear elevations, having a bone-facing surface for abutting at least a portion of the trochlea of the patient’s knee and an articular surface generally opposite the bone-facing surface; each articular surface having a curvature (J-curve) generally disposed in a first plane (sagittal plane) and ML curve generally disposed in a second and third plane (frontal plane and transverse planes); each articular surfaces of the medial and lateral elevations optionally having an offset and a depth to the trochlea in the second and third plane, which can be equivalent or not;
(c) the articular surface orientation of the trochlea portion to the distal and posterior condylar portions of the distal and posterior condyles are not dependent and are parallel or obliquely oriented (convergent or divergent) in at least one of the planes;
(d) an ML condylar offset is optionally integrated between the medial and lateral articular surfaces of the distal (::: distal condylar offset) and posterior (= posterior condylar offset) condylar portions of the distal and posterior condyles, this condylar offset being optionally equivalent between distal and posterior condylar portions; and
(e) an ML trochlear offset is optionally integrated between the medial and lateral articular surfaces of the medial and lateral trochlear elevation, this trochlear offset optionally being the same as the condylar offset of the distal and posterior condyles.

109. The prosthesis of the claim immediately above, wherein the sagittal J-curve of at least one of the joint-surface from the distal and posterior condyles (medial, lateral) or the trochlea (elevation, trochlea depth) is defined by a single, double or multi-radius or is fitting with a patient-specific J-curve.

110. The prosthesis of claim 108, wherein the sagittal J-curve of at least one of the medial and lateral joint-surfaces from the distal and posterior condyles is optionally positioned at a fixed distance to the trochlea J-curves (medial and / or lateral elevation, trochlea depth), optionally symmetrically.

111. The prosthesis of one of claims 107, wherein the sagittal J-curve of at least one of the joint-surface from the distal and posterior condyles (medial, lateral = narrowing angle) or the trochlea (medial and / or lateral elevation, trochlea depth = sulcus axis in frontal plane, Whiteside line in the axial plane) is optionally obliquely oriented in at least one of the planes, mainly the frontal and the axial planes.

112. The prosthesis of claim 107, wherein at least one of the joint facing-surface of the condylar portion and / or of the trochlea portion has an articular geometry and dimensions corresponding (or close matching, or close fitting) to the patient’s knee articular surface, in terms of sizing (comprising at least AP sizing), shape (comprising at least condylar and trochlear offset, J-curves and ML curves) and contour (comprising at least AP/ML, sizing, narrowing angle, trochlear height, posterior condyles height).

113. The prosthesis of claim 107, wherein the tangent linking the medial and lateral most distal points of the bone- facing surfaces of the distal or the tangent linking the medial and lateral most posterior points of the bone-facing surfaces of the posterior condyles or the tangent linking the medial and lateral most anterior points of the bone-facing surfaces of trochlea portion are parallel or oblique with respect to one another.

114. The prosthesis of claim 108, wherein the bone-facing surfaces are defined with a single straight flat or oblique surface or with two staggered (offset) flat or oblique surfaces or with a staggered (offset) curved surface.

115. The prosthesis of claim 108, wherein the bone-facing surfaces are fixed to the bone with optionally with a cemented fixation.

116. The prosthesis of claim 108, wherein the prosthesis corresponds to different systems selected from one of the group of systems consisting of PS: Postero-Stabilized, UC: Ultra-Congruent, PCR: Posterior Cruciate Retaining, and BCR: Bi-Cruciate Retaining, and is adapted for mobile insert or fixed insert, for primary or revision knee (semi-constrained or constrained, hinged), optionally for cemented fixation, for monobloc or modular components, and for any material typically a material selected from one of the group of materials consisting of Ti, CrCo, and Ceramic.

117. A method of manufacturing a knee prosthesis from a 3D model selected after applying a method, the method including:
(a) analysis of a patient’s current and pre-pathological knee motion behavior as well as the patient’s 3D HKA alignment,
(b) optionally using a planning algorithm, selection of a suitable 3D model from a comprehensive database of 3D models of varying knee morphologies, each 3D model being adapted to a known morphology as well as production limitations and requirements, and
(c) manufacturing of the selected 3D model which represents a producible and essentially custom knee prosthesis adapted to the individual patient’s 3D constitutional anatomy, thus making it possible to re-create the knee articulation as it was naturally.

118. A nontransient information storage medium having a knee prosthesis characterization and selection program that instructs a processor to implement the above method of claim 70 so as to accept inputs and produce outputs.

119. A nontransient information storage medium having a knee prosthesis characterization and selection program encoded thereon that, when executed, implements a method which instructs a processor to execute steps which aid a user in selecting a 3D knee prosthesis model for a particular patient, the method consisting of at least the steps of:
(a) parameterizing a knee prosthesis according to well-defined and independent knee joint compartments,
(b) generating a large number knee shapes in the form of 3D knee prosthesis models which reproduce the 3D shape asymmetries of a large number of individual knee samples including models that replicate the knee motion of essentially any patient by generating shapes which vary the shape parameters (surface and dimension) of at least one of the compartments and storing these 3D knee prosthesis models in association with each model’s shape parameters and asymmetries in a database allowing comparing of the asymmetries of the patient’s knee with those of the 3D knee prosthesis model, and
(c) after studying the patient’s pathology and developing pre-pathological knee prosthesis criteria matching the patient’s needs, optionally using a planning algorithm, searching the database comparing the large number of knee shapes based on a best match of the asymmetries of each model to identify candidate matches:
(d) providing a display of candidate matches and their attributes on an output device;
(e) providing a means of selecting a best match among suitable candidate matches identified; and
(f) optionally generating a production order if the selected knee prosthesis is not in inventory.

120. The medium of the claim immediately above, wherein the processor responds to inputs and outputs communicated by the program to and from a user.
